(19) United States
(12) Patent Application Publication
(10) Pub. No.: US 2026/0115150 A1
(43) Pub. Date: Apr. 30, 2026

(54) FORMULATIONS COMPRISING INOSITOL, SILICATES, AND OPTIONALLY AMINO ACIDS WITH IMPROVED SOLUBILITY AND METHODS OF MAKING THE SAME

(71) Applicant: Nutrition21, LLC, Saddle Brook, NJ (US)

(72) Inventor: Cara Cesario, New York, NY (US)

(21) Appl. No.: 19/371,866

(22) Filed: Oct. 28, 2025

Related U.S. Application Data
(60) Provisional application No. 63/712,812, filed on Oct. 28, 2024.

(51) Int. Cl. A61K 31/047; A23L 2/54; A23L 2/68; A61K 9/00; A61K 9/16; A61K 9/19; A61K 31/198; A61K 33/00; A61K 36/21

(52) U.S. Cl. CPC A61K 31/047; A23L 2/54; A23L 2/68; A61K 9/0095; A61K 9/1611; A61K 9/1682; A61K 9/19; A61K 31/198; A61K 33/00; A61K 36/21

(57) ABSTRACT

Provided herein are compositions comprising a silicate, inositol, and optionally an amino acid, which have improved solubility and/or are crystalline. Also provided are methods of making the disclosed compositions, as well as methods of making beverages comprising the disclosed compositions.

### CROSS-REFERENCE TO RELATED APPLICATIONS

This application claims priority to U.S. Provisional Application No. 63/712,812 filed Oct. 28, 2024, the contents of which is incorporated by reference herein in its entirety.

### BACKGROUND OF THE INVENTION

Compositions comprising silicates, inositol, and optionally amino acids can be used to provide bioavailable sources of the component ingredients. Such compositions have many health and sports nutrition benefits. In addition to increasing nitric oxide (NO) levels, and therefore blood flow, such compositions can benefit recovery, muscle damage, energy, muscle pump, cognitive function, wound healing, and bone health. Additionally, such compositions can be useful for improving the appearance of skin, hair, and nails. However, these compositions typically exhibit poor water solubility, which limits their utility in nutritional beverages. Accordingly, there is a need for alternative compositions having improved water solubility.

### SUMMARY

In certain aspects, provided herein are compositions comprising a silicate and inositol, wherein the solubility of the composition in water is at least 5 wt % at about 20° C. The compositions may further comprise an amino acid, such as arginine.

In further aspects, provided herein are compositions comprising arginine, a silicate, and inositol, wherein the solubility of the composition in water is at least 5 wt % at about 20° C.

In yet further aspects, provided herein are freeze dried compositions comprising a silicate and inositol, wherein the solubility of the composition in water is at least 5 wt % at about 20° C. The compositions may further comprise an amino acid, such as arginine.

In still further aspects, provided herein are freeze dried compositions comprising arginine, a silicate, and inositol, wherein the solubility of the composition in water is at least 5 wt % at about 20° C.

In certain embodiments, the compositions of the disclosure further comprise a hydroxide source, such as sodium hydroxide or potassium hydroxide.

In certain aspects, provided herein are methods of making the disclosed compositions comprising:
combining the silicate, the inositol, and when present, the arginine in water to form a first mixture;
optionally adding a hydroxide source to the first mixture to afford a second mixture; and
freeze drying the first mixture or the second mixture to afford the composition.

In further aspects, provided herein are methods of making the disclosed compositions comprising:
optionally, an amino acid;
a silicate; and
inositol;
wherein the solubility of the composition in water is at least 5 wt % at about 20° C.;
   the method comprising:
combining the silicate, the inositol, and when present, the arginine in water to form a first mixture;
optionally adding a hydroxide source to the first mixture to afford a second mixture; and
freeze drying the first mixture or the second mixture to afford the composition.

In yet further aspects, provided herein are methods of making a beverage comprising a composition comprising:
optionally, an amino acid;
a silicate; and
inositol;
wherein the solubility of the composition in water is at least 5 wt % at about 20° C.;
   the method comprising:
combining the silicate, the inositol, and when present, the amino acid in water to form a first mixture;
optionally adding a hydroxide source to the first mixture to afford a second mixture;
freeze drying the first mixture or the second mixture to afford the composition; and
dissolving the composition in water to afford the beverage.

In still further aspects, provided herein are beverages prepared by disclosed methods.

In certain aspects, provided herein are compositions comprising:
arginine;
a silicate; and
inositol,
wherein the composition has an X-ray powder diffraction (XRPD) pattern comprising 2-Theta peaks at 18.4±0.1°, 19.2±0.1°, and 27.5±0.1°.

### BRIEF DESCRIPTION OF THE FIGURES

FIG. 1 is a Thermogravimetric Analysis (TGA) thermogram of Composition 1.

FIG. 2 is a Differential Scanning Calorimetry (DSC) thermogram of Composition 1.

FIG. 3 is a TGA thermogram of Composition 2.

FIG. 4 is a DSC thermogram of Composition 2.

FIG. 5 is a TGA thermogram of Composition 3.

FIG. 6 is a DSC thermogram of Composition 3.

FIG. 7 is a TGA thermogram of Composition 4.

FIG. 8 is a DSC thermogram of Composition 4.

FIG. 9 is a TGA thermogram of Composition 5.

FIG. 10 is a DSC thermogram of Composition 5.

FIG. 11 is an XRPD pattern of Composition 1.

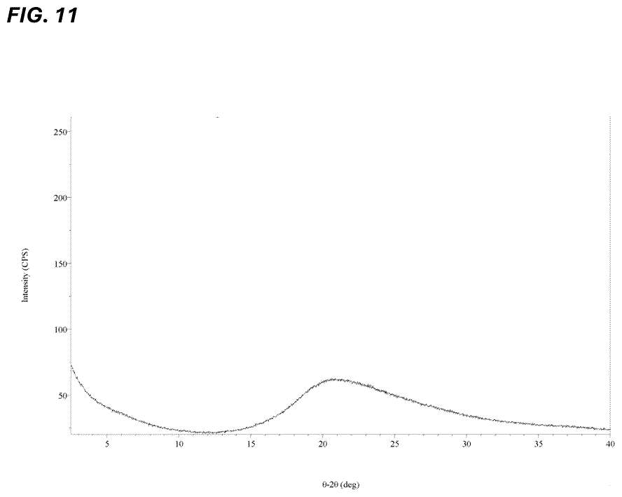

FIG. 12 is an XRPD pattern of Composition 2.

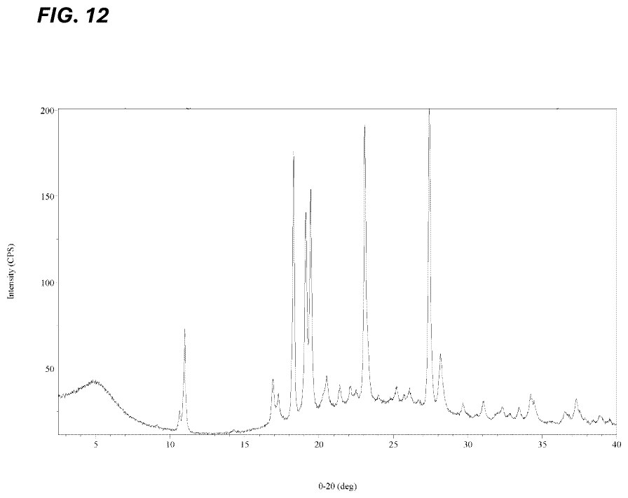

FIG. 13 is an XRPD pattern of Composition 3.

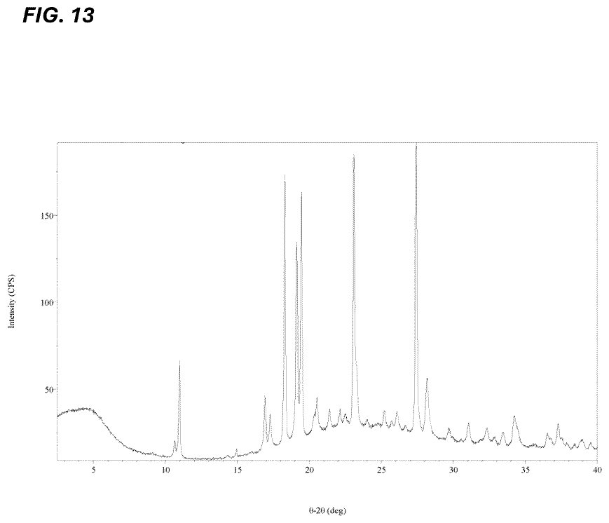

FIG. 14 depicts scanning electron microscopy (SEM) images of Composition 1.

FIG. 15 depicts SEM images of Composition 2.

FIG. 16 depicts SEM images of Composition 3.

FIG. 17 depicts SEM images of Composition 4.

FIG. 18 depicts SEM images of Composition 5.

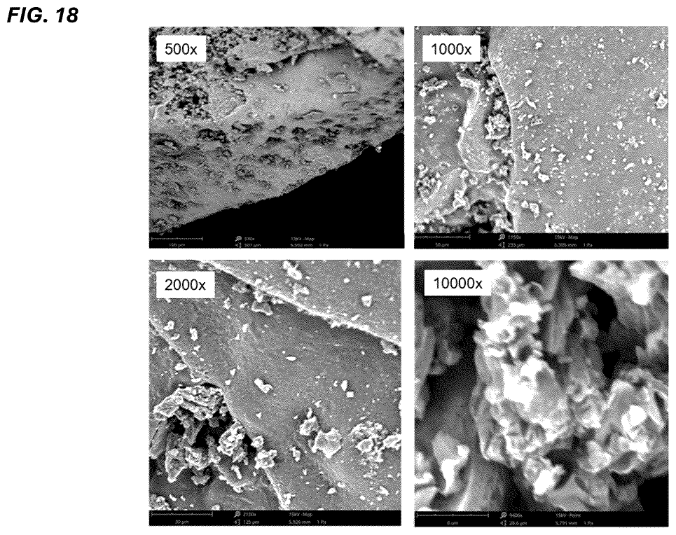

FIG. 19 depicts SEM images of Composition 8.

FIG. 20 depicts SEM images of Composition 13.

FIG. 21 is an XRPD pattern of a L-arginine reference standard.

FIG. 22 is an XRPD pattern of an inositol reference standard.

FIG. 23 is an XRPD pattern of Composition 13.

FIG. 24 is stacked plot of XRPD patterns of inositol and L-arginine with peak labels.

FIG. 25 is stacked plot of XRPD patterns of Composition 13, inositol and L-arginine.

FIG. 26 is stacked plot of XRPD patterns of Composition 13, inositol and L-arginine with the apex of the XRPD peaks for Composition 13 marked with lines to show the alignment with the reference standards.

FIG. 27 is stacked plot of XRPD patterns of Composition 13, inositol and L-arginine with peak labels.

FIG. 28 is a TGA thermogram of Composition 8.

FIG. 29 is a DSC thermogram of Composition 8.

FIG. 30 is a TGA thermogram of Composition 13.

FIG. 31 is a DSC thermogram of Composition 13.

FIG. 32 is an XRPD pattern of spray dried Composition 22.

FIG. 33 is a TGA thermogram of spray dried Composition 22.

FIG. 34 is a DSC thermogram of spray dried Composition 22.

FIG. 35 is a stacked plot of ¹H NMR spectra of Composition 21 (top), Composition 20 (middle), and spray dried Composition 22 (bottom).

FIG. 36 is a stacked plot of ¹³C NMR spectra of Composition 21 (top), Composition 20 (middle), and spray dried Composition 22 (bottom).

FIG. 37 is an overlay of the fitted Lorentzian-Gaussian curve for ²⁹Si solid-state NMR spectrum of spray dried Composition 22 and its individual deconvoluted lines.

FIG. 38 is an optical microscopy image of spray dried Composition 22.

FIG. 39 is a stacked plot of IR spectra of Spectral Sum P (top, linear combination of the spectra of arginine, inositol, and potassium silicate), spray dried Composition 22 (second from top), arginine (middle), inositol (second from bottom) and potassium silicate (bottom).

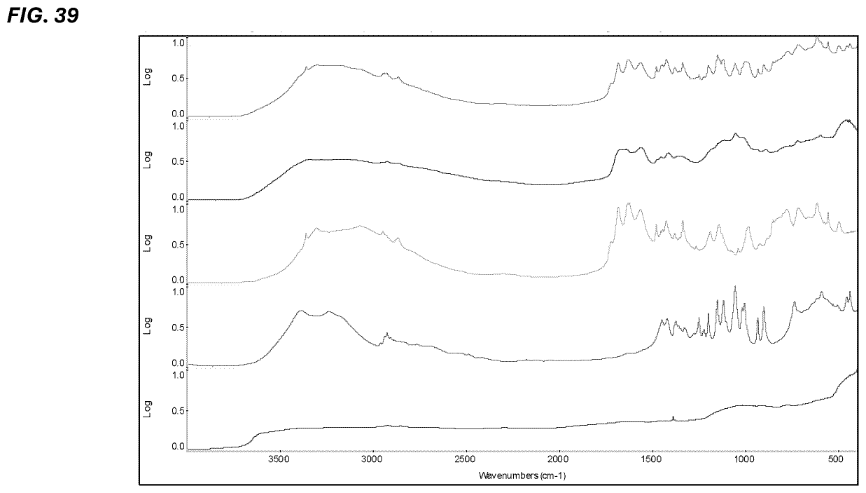

FIG. 40 is a stacked plot of IR spectra of Spectral Sum S (top, linear combination of the spectra of arginine, inositol, and sodium silicate), arginine (second from top), inositol (second from bottom), and sodium silicate (bottom).

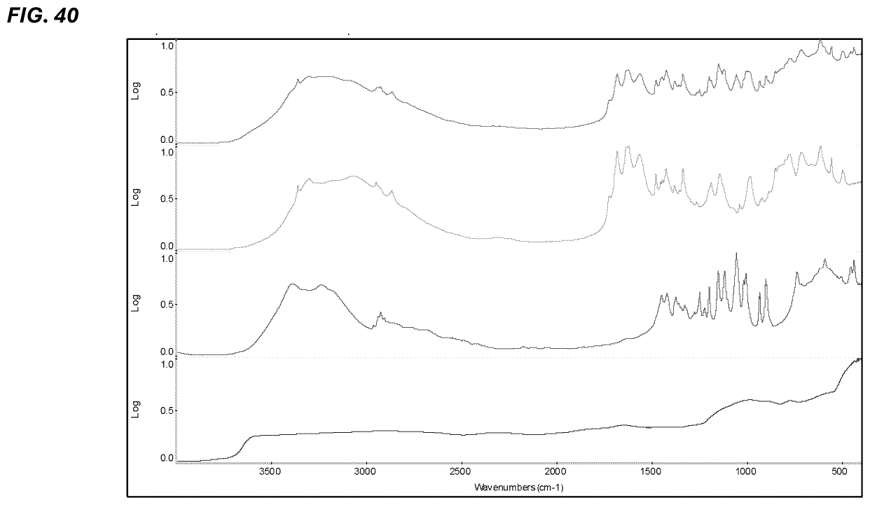

FIG. 41 is a stacked plot of the IR spectra of Physical Mixture S, Composition 21, Composition 23, Composition 24, and the individual components of spray dried Composition 22.

FIG. 42 is an XRPD pattern of Composition 21.

FIG. 43 is an XRPD pattern of Composition 20.

FIG. 44 is an IR spectrum of Composition 21.

FIG. 45 is an IR spectrum of Composition 20.

FIG. 46 is a TGA thermogram of Composition 21.

FIG. 47 is a TGA thermogram of Composition 20

FIG. 48 is a stacked plot of XRPD patterns of inositol (top), L-arginine (second from top), potassium silicate (third from top), Composition 20 (second from bottom), and Composition 21 (bottom).

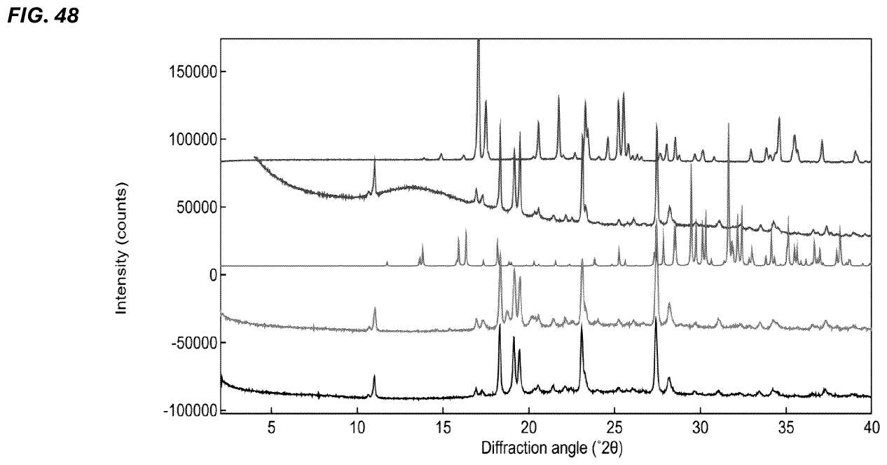

FIG. 49 is a DSC thermogram of Composition 21.

FIG. 50 is a DSC thermogram of Composition 20.

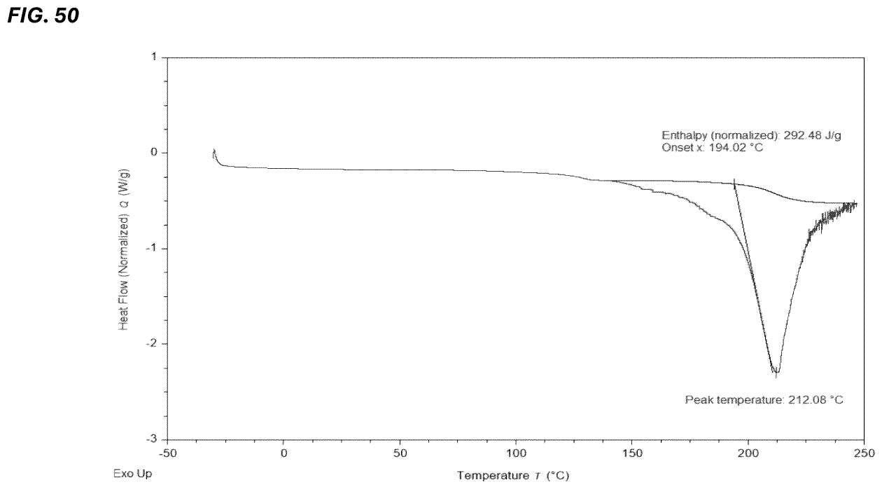

FIG. 51 is a stacked plot of ¹H NMR spectrum of Composition 21 (top) and Composition 20 (bottom).

FIG. 52 is a stacked plot of ¹³C NMR spectrum of Composition 21 (top) and Composition 20 (bottom).

FIG. 53 is an overlay of the fitted Lorentzian-Gaussian curve for ²⁹Si solid-state NMR spectrum of Composition 21 and its individual deconvoluted lines.

FIG. 54 is an overlay of the fitted Lorentzian-Gaussian curve for ²⁹Si solid-state NMR spectrum of Composition 20 and its individual deconvoluted lines.

FIG. 55 is two optical microscopy images of Composition 21.

FIG. 56 is two optical microscopy images of Composition 20.

FIG. 57 is a stacked plot of ¹H NMR spectra of (from top to bottom): Composition 22, Composition 21, Composition 23, Composition 24, Physical Mixture P, Physical Mixture S, inositol, arginine and ornithine.

FIG. 58 is an optical microscopy image of milled Composition 25.

FIG. 59 is an XRPD pattern of Composition 27.

### DETAILED DESCRIPTION

### Compositions of the Disclosure

In certain aspects, provided herein are compositions comprising a silicate and inositol, wherein the solubility of the composition in water is at least 5 wt % at about 20° C. In certain embodiments, the compositions further comprise an amino acid, preferably wherein the amino acid is arginine. In further embodiments, the composition is soluble in less than 15 minutes at 5 wt %.

In further aspects, provided herein are compositions comprising arginine, a silicate, and inositol, wherein the solubility of the composition in water is at least 5 wt % at about 20° C. In certain embodiments the composition is soluble in less than 15 minutes at 5 wt %.

In yet further aspects, provided herein are freeze dried compositions comprising a silicate and inositol, wherein the solubility of the composition in water is at least 5 wt % at about 20° C. In certain embodiments, the compositions further comprise an amino acid, preferably wherein the amino acid is arginine. In further embodiments, the composition is soluble in less than 15 minutes at 5 wt %.

In still further aspects, provided herein are freeze dried compositions comprising arginine, a silicate, and inositol, wherein the solubility of the composition in water is at least 5 wt % at about 20° C.

In certain aspects, provided herein are compositions comprising:
arginine;
a silicate; and
inositol,
wherein the composition has an X-ray powder diffraction (XRPD) pattern comprising 2-Theta peaks at 18.4±0.1°, 19.2±0.1°, and 27.5±0.1°.

In certain embodiments, the solubility of the composition in water is at least 1%. In other embodiments, the solubility of the composition is at least 5%. In other embodiments, the solubility of the composition is at least 10 wt % at about 20° C. In further embodiments, the solubility of the composition is at least 15%. In yet further embodiments, the solubility of the composition in water is at least 20 wt % at about 20° C. In still further embodiments, the solubility of the composition in water is at least 30 wt % at about 20° C. In certain embodiments, the solubility of the composition in water is at least 40 wt % at 20° C. In further embodiments, the solubility of the composition in water is at least 50 wt % at about 20° C. In yet further embodiments, the solubility of the composition in water is at least 60 wt % at about 20° C. In still further embodiments, the solubility of the composition in water is at least 70 wt % at about 20° C. In certain embodiments, the solubility of the composition in water is at least 80 wt % at about 20° C. In further embodiments, the solubility of the composition in water is at least 90 wt % at about 20° C.

In certain embodiments, the solubility of the composition in water is about 10 wt % at about 20° C. In further embodiments, the solubility of the composition in water is about 20 wt % at about 20° C. In yet further embodiments, the solubility of the composition in water is about 30 wt % at about 20° C. In still further embodiments, the solubility of the composition in water is about 40 wt % at about 20° C. In certain embodiments, the solubility of the composition in water is about 50 wt % at about 20° C. In further embodiments, the solubility of the composition in water is about 60 wt % at about 20° C. In yet further embodiments, the solubility of the composition in water is about 70 wt % at about 20° C. In still further embodiments, the solubility of the composition in water is about 80 wt % at about 20° C. In certain embodiments, the solubility of the composition in water is about 90 wt % at about 20° C.

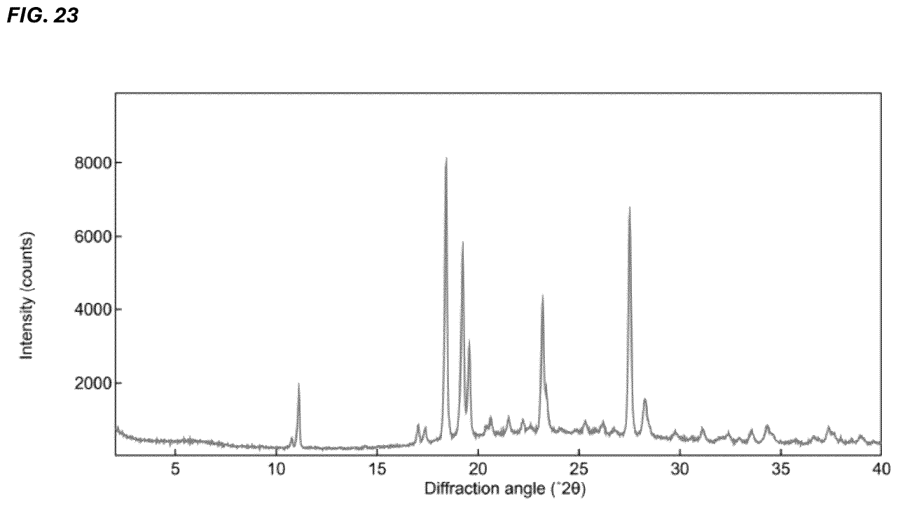

In certain embodiments, the composition has an X-ray powder diffraction (XRPD) pattern comprising 2-Theta peaks at 18.4±0.1°, 19.2±0.1°, and 27.5±0.1°. In further embodiments, the XRPD pattern further comprises a 2-Theta peak at 23.2±0.1°. In yet further embodiments, the XRPD pattern further comprises a 2-Theta peak at 11.1±0.1°. In still further embodiments, the XRPD pattern further comprises a 2-Theta peak at 19.60±0.1°. In certain embodiments, the XRPD pattern further comprises a 2-Theta peak at 17.0±0.1°. In further embodiments, the XRPD pattern further comprises a 2-Theta peak at 17.4±0.1°. In yet further embodiments, the XRPD pattern further comprises a 2-Theta peak at 28.2±0.1°. In still further embodiments, the XRPD pattern further comprises a 2-Theta peak at 21.5±0.1°. In certain embodiments, the composition has an X-ray powder diffraction (XRPD) pattern substantially the same as shown in FIG. 23.

In further embodiments, the composition has an X-ray powder diffraction (XRPD) pattern comprising additional 2-Theta peaks at about 9.41, about 11.34, about 13.60, about 15.03, about 16.09, about 16.72, about 18.73, about 22.74, about 27.72, about 28.79, about 29.42, about 29.45, about 31.68, about 32.61, and about 36.10.

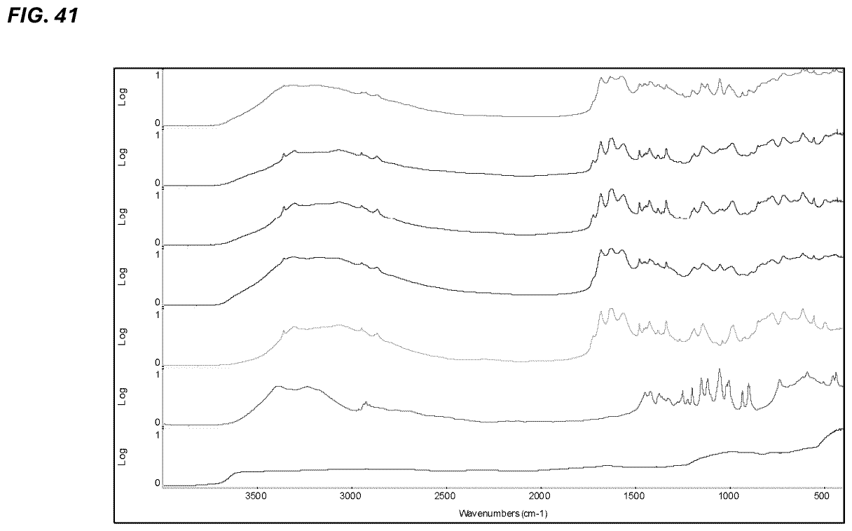

In certain embodiments, the composition has an Infrared (IR) pattern comprising peaks from about 1475 cm⁻¹ to about 1720 cm⁻¹. In certain embodiments, the composition comprises additional peaks from about 500 cm⁻¹ to about 875 cm⁻¹. In certain embodiments, the composition has an IR pattern substantially the same as shown in FIG. 41.

In certain embodiments, the composition further comprises a hydroxide source. Any suitable alkali metal hydroxide source may be used. Preferred hydroxide sources include sodium hydroxide, potassium hydroxide, or a combination thereof.

In certain preferred embodiments, the spray dried composition comprises potassium silicate. In certain preferred embodiments, the composition comprises sodium silicate. In certain preferred embodiments, the milled composition comprises sodium silicate. In certain preferred embodiments, the admixture comprises sodium silicate. In certain embodiments, the arginine, the silicate, and the inositol do not form an electrostatic or ionic complex. In certain preferred embodiments the arginine, the silicate, and the inositol form a heterogeneous composition.

The hydroxide source may be present in a molar ratio from about 0.1:1 to about 0.5:1 relative to the arginine, or about 0.15:1 to about 0.25:1 relative to the arginine. In certain embodiments, the hydroxide source is present in a molar ratio of about 0.01:1 to about 1.0:1 relative to the arginine. In further embodiments, the hydroxide source is present in a molar ratio of about 0.02:1 to about 0.9:1 relative to the arginine. In yet further embodiments, the hydroxide source is present in a molar ratio of about 0.03:1 to about 0.8:1 relative to the arginine. In still further embodiments, the hydroxide source is present in a molar ratio of about 0.04:1 to about 0.7:1 relative to the arginine.

In certain embodiments, the hydroxide source is present in a molar ratio of about 0.05:1 to about 0.6:1 relative to the arginine. In further embodiments, the hydroxide source is present in a molar ratio of about 0.06:1 to about 0.5:1 relative to the arginine. In further embodiments, the hydroxide source is present in a molar ratio of about 0.07:1 to about 0.5:1 relative to the arginine. In still further embodiments, the hydroxide source is present in a molar ratio of about 0.08:1 to about 0.5:1 relative to the arginine.

In certain embodiments, the hydroxide source is present in a molar ratio of, about 0.09:1 to about 0.5:1 relative to the arginine. In further embodiments, the hydroxide source is present in a molar ratio of about 0.1:1 to about 0.4:1 relative to the arginine. In yet further embodiments, the hydroxide source is present in a molar ratio of about 0.1:1 to about 0.3:1 relative to the arginine. In still further embodiments, the hydroxide source is present in a molar ratio of about 0.1:1 to about 0.2:1 relative to the arginine.

In certain embodiments, provided herein are admixtures comprising:
arginine;
sodium silicate;
sodium hydroxide; and
inositol,
wherein the admixture has a Particle Size Distribution (PSD) d₁₀ value of about 20 μm to about 23 μm, a d₅₀ value of about 114 μm to about 122 μm, and/or a d₉₀ value of about 390 μm to about 433 μm.

In certain embodiments, provided herein are milled compositions comprising:
arginine;
sodium silicate;
sodium hydroxide; and
inositol,
wherein the milled composition has a Particle Size Distribution (PSD) d₁₀ value of about 22 μm to about 28 μm, a d₅₀ value of about 185 μm to about 193 μm, and/or a d₉₀ value of about 553 μm to about 581 μm.

In certain preferred embodiments, the arginine, sodium silicate, and sodium hydroxide of the milled composition do not form an electrostatic or ionic complex. In certain preferred embodiments, the arginine, sodium silicate, and sodium hydroxide of the milled composition form a heterogeneous composition.

In certain preferred embodiments, the arginine, sodium silicate, and inositol of the admixture do not form an electrostatic or ionic complex. In certain preferred embodiments, the arginine, sodium silicate, and inositol of the admixture form a heterogeneous composition.

In certain embodiments, the compositions do not include a hydroxide source.

In some embodiments, the molar ratio of arginine to silicate is about 0.01:1 to about 2.0:1. In further embodiments, the hydroxide source is present in a molar ratio of about 0.01:1 to about 1.9:1. In still further embodiments, the hydroxide source is present in a molar ratio of about 0.02:1 to about 1.8:1. In further embodiments, the hydroxide source is present in a molar ratio of about 0.03:1 to about 1.7:1. In further embodiments, the hydroxide source is present in a molar ratio of about 0.04:1 to about 1.6:1. In yet further embodiments, the hydroxide source is present in a molar ratio of about 0.05:1 to about 1.5:1. In further embodiments, the hydroxide source is present in a molar ratio of about 0.06:1 to about 1.4:1. In further embodiments, the hydroxide source is present in a molar ratio of about 0.07:1 to about 1.3:1. In further embodiments, the hydroxide source is present in a molar ratio of about 0.08:1 to about 1.2:1. In further embodiments, the hydroxide source is present in a molar ratio of about 0.09:1 to about 1.1:1. In some preferred embodiments, the molar ratio of arginine to silicate is about 0.5:1 to about 2:1, preferably about 0.75:1 to about 1.25:1, more preferably about 0.8:1 to about 1.2:1, and more preferable about 1:1.

In some embodiments, the molar ratio of the inositol to arginine is about 0.01:1 to about 2.0:1. In further embodiments, the hydroxide source is present in a molar ratio of about 0.01:to about 1.9:1. In further embodiments, the hydroxide source is present in a molar ratio of about 0.02:1 to about 1.8:1. In yet further embodiments, the hydroxide source is present in a molar ratio of about 0.03:1 to about 1.7:1, about 0.04:1 to about 1.6:1, about 0.05:1 to about 1.5:1, about 0.06:1 to about 1.4:1, about 0.07:1 to about 1.3:1, about 0.08:1 to about 1.2:1, about 0.09:1 to about 1.1:1, about 0.1:1 to about 1:1, about 0.2:1 to about 1.1:1, about 0.3:1 to about 1:1, about 0.4:1 to about 1:1. In certain preferred embodiments, the molar ratio of inositol to arginine is about 0.5:1

In some embodiments, the composition is spray dried, while in other embodiments, the composition is freeze dried. In other embodiments, the composition is an aqueous solution. In preferred embodiments, the composition is freeze dried.

In certain embodiments, provided herein is a beverage composition comprising the spray dried composition, the freeze dried composition, the milled composition, or the admixture of the present disclosure.

In certain embodiments, provided herein is a beverage composition, wherein the pH of the beverage is from about 2 to about 4. In further embodiments, provided herein is a beverage composition, wherein the pH of the beverage is from about 3 to about 4. In certain embodiments, provided herein is a beverage composition, wherein the pH of the beverage is about 2. In further embodiments, provided herein is a beverage composition, wherein the pH of the beverage is about 3. In yet further embodiments, provided herein is a beverage composition, wherein the pH of the beverage is about 4. In certain embodiments, the pH of the beverage is adjusted with citric acid. In other embodiments, the pH of the beverage is adjusted with malic acid. In certain embodiments, the beverage is carbonated. In other embodiments, the beverage is non-carbonated. In further embodiments, the beverage comprises an additional ingredient selected from a nitrate or a salt thereof, beetroot, beetroot extract, or a combination thereof.

Silicates that are useful in the present compositions include potassium silicate, sodium silicate, or any combination thereof. In some embodiments, the silicate is potassium silicate, sodium silicate, or a combination thereof.

### Particle Size Distribution for Milled Compositions and Admixtures

In certain embodiments, the milled composition has a diameter distribution characterized by a d₁₀ of about 22 μm to about 28 μm. In further embodiments, the milled composition has a diameter distribution characterized by a d₁₀ of about 23 μm to about 27 μm. In certain embodiments, the milled composition has a diameter distribution characterized by a d₁₀ of about 23 μm. In further embodiments, the milled composition has a diameter distribution characterized by a d₁₀ of about 24 μm. In yet further embodiments, the milled composition has a diameter distribution characterized by a d₁₀ of about 25 μm. In still further embodiments, the milled composition has a diameter distribution characterized by a d₁₀ of about 26 μm. In certain embodiments, the milled composition has a diameter distribution characterized by a d₁₀ of about 27 μm.

In certain embodiments, the milled composition has a diameter distribution characterized by a d₅₀ of about 185 μm to about 193 μm. In further embodiments, the milled composition has a diameter distribution characterized by a d₅₀ of about 186 μm to about 192 μm. In certain embodiments, the milled composition has a diameter distribution characterized by a d₅₀ of about 186 μm. In further embodiments, the milled composition has a diameter distribution characterized by a d₅₀ of about 187 μm. In yet further embodiments, the milled composition has a diameter distribution characterized by a d₅₀ of about 188 μm. In still further embodiments, the milled composition has a diameter distribution characterized by a d₅₀ of about 189 μm. In certain embodiments, the milled composition has a diameter distribution characterized by a d₅₀ of about 190 μm. In further embodiments, the milled composition has a diameter distribution characterized by a d₅₀ of about 191 μm. In yet further embodiments, the milled composition has a diameter distribution characterized by a d₅₀ of about 192 μm.

In certain embodiments, the milled composition has a diameter distribution characterized by a d₉₀ of about 553 μm to about 581 μm. In further embodiments, the milled composition has a diameter distribution characterized by a d₉₀ of about 554 μm to about 580 μm. In certain embodiments, the milled composition has a diameter distribution characterized by a d₉₀ of about 554 μm. In further embodiments, the milled composition has a diameter distribution characterized by a d₉₀ of about 555 μm. In yet further embodiments, the milled composition has a diameter distribution characterized by a d₉₀ of about 556 μm. In still further embodiments, the milled composition has a diameter distribution characterized by a d₉₀ of about 557 μm. In certain embodiments, the milled composition has a diameter distribution characterized by a d₉₀ of about 558 μm. In further embodiments, the milled composition has a diameter distribution characterized by a d₉₀ of about 559 μm. In yet further embodiments, the milled composition has a diameter distribution characterized by a d₉₀ of about 560 μm. In still further embodiments, the milled composition has a diameter distribution characterized by a d₉₀ of about 561 μm. In certain embodiments, the milled composition has a diameter distribution characterized by a d₉₀ of about 562 μm. In further embodiments, the milled composition has a diameter distribution characterized by a d₉₀ of about 563 μm. In yet further embodiments, the milled composition has a diameter distribution characterized by a d₉₀ of about 564 μm. In still further embodiments, the milled composition has a diameter distribution characterized by a d₉₀ of about 565 μm. In certain embodiments, the milled composition has a diameter distribution characterized by a d₉₀ of about 566 μm. In further embodiments, the milled composition has a diameter distribution characterized by a d₉₀ of about 567 μm. In yet further embodiments, the milled composition has a diameter distribution characterized by a d₉₀ of about 568 μm. In still further embodiments, the milled composition has a diameter distribution characterized by a d₉₀ of about 569 μm. In certain embodiments, the milled composition has a diameter distribution characterized by a d₉₀ of about 570 μm. In further embodiments, the milled composition has a diameter distribution characterized by a d₉₀ of about 571 μm. In yet further embodiments, the milled composition has a diameter distribution characterized by a d₉₀ of about 572 μm. In still further embodiments, the milled composition has a diameter distribution characterized by a d₉₀ of about 573 μm. In certain embodiments, the milled composition has a diameter distribution characterized by a d₉₀ of about 574 μm. In further embodiments, the milled composition has a diameter distribution characterized by a d₉₀ of about 575 μm. In yet further embodiments, the milled composition has a diameter distribution characterized by a d₉₀ of about 576 μm. In still further embodiments, the milled composition has a diameter distribution characterized by a d₉₀ of about 577 μm. In certain embodiments, the milled composition has a diameter distribution characterized by a d₉₀ of about 578 μm. In further embodiments, the milled composition has a diameter distribution characterized by a d₉₀ of about 579 μm. In yet further embodiments, the milled composition has a diameter distribution characterized by a d₉₀ of about 580 μm.

In certain embodiments, the admixture has a diameter distribution characterized by a d₁₀ of about 20 μm to about 23 μm. In further embodiments, the admixture has a diameter distribution characterized by a d₁₀ of about 21 μm to about 22 μm. In certain embodiments, the admixture has a diameter distribution characterized by a d₁₀ of about 21 μm. In further embodiments, the admixture has a diameter distribution characterized by a d₁₀ of about 22 μm.

In certain embodiments, the admixture has a diameter distribution characterized by a d₅₀ of about 114 μm to about 122 μm. In further embodiments, the admixture has a diameter distribution characterized by a d₅₀ of about 115 μm to about 121 μm. In certain embodiments, the admixture has a diameter distribution characterized by a d₅₀ of about 115 μm. In further embodiments, the admixture has a diameter distribution characterized by a d₅₀ of about 116 μm. In yet further embodiments, the admixture has a diameter distribution characterized by a d₅₀ of about 117 μm. In still further embodiments, the admixture has a diameter distribution characterized by a d₅₀ of about 118 μm. In certain embodiments, the admixture has a diameter distribution characterized by a d₅₀ of about 119 μm. In further embodiments, the admixture has a diameter distribution characterized by a d₅₀ of about 120 μm. In yet further embodiments, the admixture has a diameter distribution characterized by a d₅₀ of about 121 μm.

In certain embodiments, the admixture has a diameter distribution characterized by a d₉₀ of about 390 μm to about 433 μm. In further embodiments, the admixture has a diameter distribution characterized by a d₉₀ of about 391 μm to about 432 μm. In certain embodiments, the admixture has a diameter distribution characterized by a d₉₀ of about 391 μm. In further embodiments, the admixture has a diameter distribution characterized by a d₉₀ of about 392 μm. In yet further embodiments, the admixture has a diameter distribution characterized by a d₉₀ of about 393 μm. In still further embodiments, the admixture has a diameter distribution characterized by a d₉₀ of about 394 μm. In certain embodiments, the admixture has a diameter distribution characterized by a d₉₀ of about 395 μm. In further embodiments, the admixture has a diameter distribution characterized by a d₉₀ of about 396 μm. In yet further embodiments, the admixture has a diameter distribution characterized by a d₉₀ of about 397 μm. In still further embodiments, the admixture has a diameter distribution characterized by a d₉₀ of about 398 μm. In certain embodiments, the admixture has a diameter distribution characterized by a d₉₀ of about 399 μm. In further embodiments, the admixture has a diameter distribution characterized by a d₉₀ of about 400 μm. In yet further embodiments, the admixture has a diameter distribution characterized by a d₉₀ of about 401 μm. In still further embodiments, the admixture has a diameter distribution characterized by a d₉₀ of about 402 μm. In certain embodiments, the admixture has a diameter distribution characterized by a d₉₀ of about 403 μm. In further embodiments, the admixture has a diameter distribution characterized by a d₉₀ of about 404 μm. In yet further embodiments, the admixture has a diameter distribution characterized by a d₉₀ of about 405 μm. In still further embodiments, the admixture has a diameter distribution characterized by a d₉₀ of about 406 μm. In certain embodiments, the admixture has a diameter distribution characterized by a d₉₀ of about 407 μm. In further embodiments, the admixture has a diameter distribution characterized by a d₉₀ of about 408 μm. In yet further embodiments, the admixture has a diameter distribution characterized by a d₉₀ of about 409 μm. In still further embodiments, the admixture has a diameter distribution characterized by a d₉₀ of about 410 μm. In certain embodiments, the admixture has a diameter distribution characterized by a d₉₀ of about 411 μm. In further embodiments, the admixture has a diameter distribution characterized by a d₉₀ of about 412 μm. In yet further embodiments, the admixture has a diameter distribution characterized by a d₉₀ of about 413 μm. In still further embodiments, the admixture has a diameter distribution characterized by a d₉₀ of about 414 μm. In certain embodiments, the admixture has a diameter distribution characterized by a d₉₀ of about 415 μm. In further embodiments, the admixture has a diameter distribution characterized by a d₉₀ of about 416 μm. In yet further embodiments, the admixture has a diameter distribution characterized by a d₉₀ of about 417 μm. In still further embodiments, the admixture has a diameter distribution characterized by a d₉₀ of about 418 μm. In certain embodiments, the admixture has a diameter distribution characterized by a d₉₀ of about 419 μm. In further embodiments, the admixture has a diameter distribution characterized by a d₉₀ of about 420 μm. In yet further embodiments, the admixture has a diameter distribution characterized by a d₉₀ of about 421 μm. In still further embodiments, the admixture has a diameter distribution characterized by a d₉₀ of about 422 μm. In certain embodiments, the admixture has a diameter distribution characterized by a d₉₀ of about 423 μm. In further embodiments, the admixture has a diameter distribution characterized by a d₉₀ of about 424 μm. In yet further embodiments, the admixture has a diameter distribution characterized by a d₉₀ of about 425 μm. In still further embodiments, the admixture has a diameter distribution characterized by a d₉₀ of about 426 μm. In certain embodiments, the admixture has a diameter distribution characterized by a d₉₀ of about 427 μm. In further embodiments, the admixture has a diameter distribution characterized by a d₉₀ of about 428 μm. In yet further embodiments, the admixture has a diameter distribution characterized by a d₉₀ of about 429 μm. In still further embodiments, the admixture has a diameter distribution characterized by a d₉₀ of about 430 μm. In certain embodiments, the admixture has a diameter distribution characterized by a d₉₀ of about 431 μm. In further embodiments, the admixture has a diameter distribution characterized by a d₉₀ of about 432 μm.

### Methods of Making

In certain aspects, provided herein are methods of making the compositions of the disclosure, the method comprising:
combining the silicate, the inositol, and when present, the arginine in water to form a first mixture;
optionally adding a hydroxide source to the first mixture to afford a second mixture; and
freeze drying the first mixture or the second mixture to afford the composition.

In further aspects, provided herein are methods of making a composition comprising:
optionally, an amino acid;
a silicate; and
inositol;
wherein the solubility of the composition in water is at least 5 wt % at about 20° C.;
   the method comprising:
combining the silicate, the inositol, and when present, the amino acid in water to form a first mixture;
optionally adding a hydroxide source to the first mixture to afford a second mixture; and
freeze drying the first mixture or the second mixture to afford the composition.

In yet further aspects, provided herein are methods of making the milled compositions of the disclosure, the method comprising:
combining the silicate, the inositol, and when present, the arginine in water to form a first mixture;
optionally adding a hydroxide source to the first mixture to afford a second mixture;
freeze drying the first mixture or the second mixture to afford the composition; and
further milling the composition to produce a milled composition.

In still further aspects, provided herein are methods of making the admixture of the disclosure, the method comprising:
combining the silicate, the inositol, and when present, the arginine in water to form a first mixture;
optionally adding a hydroxide source to the first mixture to afford a second mixture;
freeze drying the first mixture or the second mixture to afford the composition;
further milling the composition to produce the milled composition; and
further adding additional inositol to the milled composition to produce an admixture.

In certain aspects, provided herein are methods of making a beverage comprising the compositions of the disclosure. In certain aspects, the composition comprises:
optionally, an amino acid;
a silicate; and
inositol;
wherein the solubility of the composition in water is at least 5 wt % at about 20° C.;
   and the method comprises:
combining the silicate, the inositol, and when present, the amino acid in water to form a first mixture;
optionally adding a hydroxide source to the first mixture to afford a second mixture;
freeze drying the first mixture or the second mixture to afford the composition; and
dissolving the composition in water to afford the beverage.

In further aspects, provided herein are beverages made by the disclosed methods. In further aspects, provided herein are methods of making beverages comprising combining any one of the disclosed compositions with water to produce the beverage.

In some embodiments, the beverage comprises from about 1 to about 25 mg/mL of the spray dried composition, the freeze dried composition, the milled composition, or the admixture of the present disclosure. In other embodiments, the beverage comprises from about 1 to about 20 mg/mL of the spray dried composition, the freeze dried composition, the milled composition, or the admixture of the present disclosure. In other embodiments, the beverage comprises from about 1 to about 15 mg/mL of the spray dried composition, the freeze dried composition, the milled composition, or the admixture of the present disclosure. In some embodiments, the beverage comprises from about 1 to about 10 mg/mL of the spray dried composition, the freeze dried composition, the milled composition, or the admixture of the present disclosure. In yet other embodiments, the beverage comprises from about 1 to about 5 mg/mL of the spray dried composition, the freeze dried composition, the milled composition, or the admixture of the present disclosure.

In more particular embodiments, the beverage comprises about 1 mg/mL of the spray dried composition, the freeze dried composition, the milled composition, or the admixture of the present disclosure. In other embodiments, the beverage comprises about 2 mg/mL of the spray dried composition, the freeze dried composition, the milled composition, or the admixture of the present disclosure. In other embodiments, the beverage comprises about 3 mg/mL of the spray dried composition, the freeze dried composition, the milled composition, or the admixture of the present disclosure. In other embodiments, the beverage comprises about 4 mg/mL of the spray dried composition, the freeze dried composition, the milled composition, or the admixture of the present disclosure. In other embodiments, the beverage comprises about 5 mg/mL of the spray dried composition, the freeze dried composition, the milled composition, or the admixture of the present disclosure. In other embodiments, the beverage comprises about 7 mg/mL of the spray dried composition, the freeze dried composition, the milled composition, or the admixture of the present disclosure. In other embodiments, the beverage comprises about 7 mg/mL of the spray dried composition, the freeze dried composition, the milled composition, or the admixture of the present disclosure. In other embodiments, the beverage comprises about 8 mg/mL of the spray dried composition, the freeze dried composition, the milled composition, or the admixture of the present disclosure. In other embodiments, the beverage comprises about 9 mg/mL of the spray dried composition, the freeze dried composition, the milled composition, or the admixture of the present disclosure. In other embodiments, the beverage comprises about 10 mg/mL of the spray dried composition, the freeze dried composition, the milled composition, or the admixture of the present disclosure.

In certain embodiments provided herein are beverages having a volume from about 1 oz to about 16 oz. In certain embodiments, the volume of the beverage is 1 oz. In further embodiments, the volume of the beverage is 2 oz. In yet further embodiments, the volume of the beverage is 3 oz. In still further embodiments, the volume of the beverage is 4 oz. In certain embodiments, the volume of the beverage is 5 oz. In further embodiments, the volume of the beverage is 6 oz. In yet further embodiments, the volume of the beverage is 7 oz. In still further embodiments, the volume of the beverage is 8 oz. In certain embodiments, the volume of the beverage is 9 oz. In further embodiments, the volume of the beverage is 10 oz. In yet further embodiments, the volume of the beverage is 11 oz. In still further embodiments, the volume of the beverage is 12 oz. In certain embodiments, the volume of the beverage is 13 oz. In further embodiments, the volume of the beverage is 14 oz. In further embodiments, the volume of the beverage is 15 oz. In still further embodiments, the volume of the beverage is 16 oz.

### Methods of Improving NO Cellular Production and Blood Flow

In certain aspects, provided herein is a method of decreasing insulin resistance in an individual is provided, the method comprising identifying an individual in need of decreased insulin resistance and administering to the individual an effective amount of the spray dried composition, the freeze dried composition, the milled composition, or the admixture of the present disclosure. In certain embodiments, the method includes administering the spray dried composition, the freeze dried composition, the milled composition, or the admixture of the present disclosure parenterally or orally. In further embodiments, the method includes administering an effective amount of the spray dried composition, the freeze dried composition, the milled composition, or the admixture between about 2 mg/kg body weight and about 2,500 mg/kg body weight. In yet further embodiments, the method includes administering an effective amount of the spray dried composition, the freeze dried composition, the milled composition, or the admixture of the present disclosure between about 5 mg/kg body weight and about 1,000 mg/kg body weight.

In certain aspects, a method for increasing nitric oxide production in an individual is provided, the method comprising administering to the individual an effective amount of the spray dried composition, the freeze dried composition, the milled composition, or the admixture of the present disclosure. In further embodiments, the method includes administering the spray dried composition, the freeze dried composition, the milled composition, or the admixture of the present disclosure parenterally or orally. In yet further embodiments, the method includes administering an effective amount of the spray dried composition, the freeze dried composition, the milled composition, or the admixture of the present disclosure between about 2 mg/kg body weight and about 2,500 mg/kg body weight.

In certain aspects, provided herein is a method for treating a disorder caused by or exacerbated by reduced levels of nitric oxide by increasing concentrations of nitric oxide in an individual is provided, the method comprising identifying an individual in need of an increased concentration of nitric oxide and administering to the individual an effective amount of the spray dried composition, the freeze dried composition, the milled composition, or the admixture. In certain embodiments, the method includes that the disorder is selected from the group consisting of pulmonary hypertension, renal disease, hypertension, diabetes, hypercholesterolemia, hyperglycemia, heart failure, Fabry's disease, chronic obstructive pulmonary disease (COPD), inflammatory bowel disease, Crohn's disease, ulcerative colitis, perinatal asphyxia, meconium aspiration syndrome, Group B Strep sepsis, congenital diaphragmatic hernia, ischemic heart disease, hyperhomocysteinemia, multiple sclerosis, Takayasu's arteritis, autosomal dominant polycystic kidney disease, end-stage renal failure, and liver disease.

In yet further embodiments, the method further comprises administering a second beneficial agent effective in treating a disorder caused by or exacerbated by reduced levels of nitric oxide, wherein the second beneficial agent is a conventional therapy for nitric oxide deficiencies. In still further embodiments, the method includes administering an effective amount of the spray dried composition, the freeze dried composition, the milled composition, or the admixture between about 2 mg/kg body weight and about 2,500 mg/kg body weight. An additional aspect of the invention is a method of reducing markers of poor cardiovascular health, preferably urinary albumin concentration or vascular contractility, in an individual, preferably a mammal, more preferably a human, comprising the step of administering to the individual an effective amount of the spray dried composition, the freeze dried composition, the milled composition, or the admixture of the present disclosure. Additional aspects of the invention include methods for improving bradykinin response, coronary blood flow, improving vascular health and reducing atherosclerotic plaques in an individual, preferably a mammal, more preferably a human, comprising the step of administering to the individual an effective amount of the spray dried composition, the freeze dried composition, the milled composition, or the admixture of the present disclosure. A further aspect of the invention is a method of improving markers of good cardiovascular health, preferably vascular relaxation, in an individual, preferably a mammal, comprising the step of administering to the individual an effective amount of the spray dried composition, the freeze dried composition, the milled composition, or the admixture of the present disclosure.

### Method for Improving Cognitive Function

In certain aspects, provided herein is a method of improving cognitive functioning in a human comprising identifying a human in need of improved cognitive functioning; and administering an amount of the spray dried composition, the freeze dried composition, the milled composition, or the admixture of the present disclosure effective to improve cognitive functioning in the human.

In certain embodiments, identifying comprises a mentally fatigued human and/or a physically fatigued human.

In further embodiments, identifying comprises administering one or more cognitive tests. In yet further embodiments, identifying includes a diagnosis of ADHD.

In yet further embodiments, the amount of administered spray dried composition, freeze dried composition, milled composition, or admixture of the present disclosure is between about 0.5 g to 5 g per day. In still further embodiments, the amount of administered dried composition, freeze dried composition, milled composition, or admixture is about 1,500 mg per day.

In certain embodiments, administering comprises self-administering a composition by mouth, the composition having an amount of the spray dried composition, the freeze dried composition, the milled composition, or the admixture of the present disclosure that is about 1,500 mg. In further embodiments, administering comprises an amount of a chromium containing composition.

In certain aspects, provided herein is a method of improving cognitive function comprising identifying a cognitive disorder in a subject; and administering a therapeutically effective amount of the spray dried composition, the freeze dried composition, the milled composition, or the admixture of the present disclosure to the subject.

In certain embodiments, identifying includes administering a test that is sensitive to detecting one or more cognitive disorders. In yet further embodiments, the test is sensitive to detecting a disease or disorder selected from the group consisting of one or more of stroke, anoxic brain injury traumatic brain injury, dementia, Alzheimer's disease, Parkinson's disease, Mild Cognitive Impairment (MCI), and age-related memory loss. In yet further embodiments, identifying includes a diagnosis of ADHD. In certain embodiments, identifying includes identifying one or more genetic defects in the subject.

In certain embodiments, administering comprises orally administering a composition containing a therapeutically effective amount of the spray dried composition, the freeze dried composition, the milled composition, or the admixture of the present disclosure. In further embodiments, the therapeutically effective amount is between about 0.5 gram per day to 5 grams per day.

In certain aspects, provided herein is a method for improving cognitive function comprising: administering a first cognitive evaluation to a subject; determining the presence of a cognitive disorder in said subject; administering a therapeutically effective amount of the spray dried composition, the freeze dried composition, the milled composition, or the admixture of the present disclosure; and administering a second cognitive evaluation on said subject, wherein said second cognitive evaluation is improved relative to said first cognitive evaluation.

In certain embodiments, determining the presence of a cognitive deficit comprises determining whether the subject has a condition selected from stroke, anoxic brain injury, traumatic brain injury, dementia, Alzheimer's disease, Parkinson's disease, MCI, and age-related memory loss, or a combination of the foregoing.

In further embodiments, the first and second cognitive evaluations are independently selected from the Automatic Trail making Test A; the Automatic Trail making Test B; Profile of Mood States; Modified Mini-Mental State Examination; Three Word Recall; 7-Minute Screen; AB Cognitive Screen; Addenbrooke's Cognitive Examination (Revised); Abbreviated Mental Test; Brief Alzheimer Screen; Brief Cognitive Scale; Cognitive Abilities Screening Instrument; Cognitive Assessment Screening Test; Cognitive Capacity Screening Examination; Clock Drawing Test; DemTect; Dementia Questionnaire; General Practitioner Assessment of Cognition; Hopkins Verbal Learning Test; Informant Questionnaire on Cognitive Decline in the Elderly; Informant Questionnaire on Cognitive Decline in the Elderly—Short Form; Minnesota Cognitive Acuity Screen; Mini-Cog; Memory Impairment Screen; Mini-Mental State Examination; Mont Montpellier Screen; Neurobehavioral Cognitive Status Examination; Rotterdam Version of the Cambridge Cognitive Examination; Rapid Dementia Screening Test; Short and Sweet Screening Instrument; Symptoms of Dementia Screener; Six Item Screener; Short Memory Questionnaire; Short Orientation Memory Concentration Test; Short Portable Mental Status Questionnaire; Short Test of Mental State; Time and Change; Telephone Interview of Cognitive Status-Modified; Verbal Fluency; Modified WORLD Test, or a combination of the foregoing.

In yet further embodiments, the therapeutically effective amount is between about 0.5 gram per day to about 5 grams per day.

### Methods of Improving the Appearance of Skin, Hair and Nails

In further aspects, provided herein is a method of improving the appearance of a subject's skin, hair, or nails comprising administering a composition of the invention. In some embodiments, the method improves the appearance of a subject's skin. In certain embodiments, the method improves the appearance of a subject's hair. According to one or more embodiments, the method improves the appearance of a subject's nails. In certain embodiments, the method improves the appearance of a subject's skin and nails, skin and hair, or hair and nails. According to one or more embodiments, the method improves the appearance of a subject's skin, hair, and nails.

In some embodiments, the method improves skin elasticity, skin hydration, skin texture (e.g., reduces skin roughness or skin scaling), facial wrinkle depth, skin inflammation, hair pigmentation, hair thickness, hair density, hair volume, hair shine, hair strength, nail strength, nail growth, hair growth, or a combination thereof.

In certain embodiments, the method increases hair density. In some embodiments, the method increases the duration of the anagen stage of hair growth. According to one or more embodiments, the method increases anagen ratio. In certain embodiments, the method decreases telogen ratio.

In further aspects, provided herein is a method of treating or preventing a condition in a subject comprising administering a composition of the invention to the subject.

In some embodiments, the condition comprises deterioration of the subject's hair, skin, or nails. In certain embodiments, the condition arises from a disease. In some embodiments, the condition comprises a symptom of a disease. In certain embodiments, the condition comprises a side effect from a treatment for a disease. In certain embodiments, the condition is a genetic condition, menopause, chronic stress, aging, or chronic inflammation. In some embodiments, the condition comprises a side effect of a medication. In certain embodiments, the condition comprises a side effect of a medication administered to treat cancer, depression, or heart disease.

In some embodiments, the condition comprises hair thinning, hair loss, skin wrinkles, spontaneously aged skin, photodamaged skin, photoaged skin, irregular skin pigmentation, or nail brittleness.

In certain embodiments, the condition comprises spontaneously aged skin. Internal skin aging, termed ‘spontaneous aging,’ is the physiological changing of the skin affected by genetic factors and occurs naturally over time.

In certain embodiments, the condition comprises photoaged skin. Photoaging from chronic ultraviolet (UV) exposure leads to a complex skin-changing process that occurs predominantly on cutaneous surfaces exposed to the sun. Photoaging mechanisms include free radicals, which lead to the accumulation of reactive oxygen species (ROS). ROS mediates harmful post-translational properties on aging skin by direct chemical alterations to DNA, cell lipids, and dermal matrix proteins, including collagens. All these processes result in an irregular and non-functional accumulation of elastic fibers in the skin. Clinical manifestations of photoaging include wrinkles, telangiectasias, laxity, loss of translucency, and various pigmented spots such as freckles and solar lentigines. Similar to photoaging, chronic exposure to solar radiation causes multiple skin disorders, including sunburn, irregular pigmentation, and skin cancer, specifically non-melanoma skin cancers.

In certain embodiments, the condition comprises photodamaged skin caused by exposure to UVB radiation. UV radiation includes UVA and UVB radiation, and without being bound by any particular theory, it is believed that UVB radiation is primarily responsible for degrading skin health, including damage from sunrays such as photoaging, wrinkles, sunburns, and skin cancers. In some embodiments, the methods described herein alleviate macroscopic skin damage from UV exposure. In certain embodiments, the methods described herein alleviate histopathological skin damage from UV exposure.

In certain embodiments, the composition is administered orally to the subject.

In some embodiments, the composition is administered topically to the subject.

In some embodiments, the composition comprises an additional active ingredient. In some embodiments, the additional active ingredient is biotin.

In some embodiments, the composition is conjunctively administered with a second composition. In some embodiments, the second composition is formulated to be administered through a different route than the composition. In some embodiments, the composition is administered orally and the second composition, such as a composition comprising biotin, is conjunctively administered topically to the subject. In certain embodiments, the composition is administered topically and the second composition is conjunctively administered orally to the subject.

In some embodiments, the biotin is provided as a salt, such as a sodium, potassium, magnesium, or calcium salt, or a combination thereof. In some embodiments, a daily dosage of the biotin is up to 10 mg, up to 9 mg, up to 8 mg, up to 7 mg, up to 6 mg, up to 5 mg, up to 4 mg, up to 3 mg, up to 2 mg, or up to 1 mg. In some embodiments, a daily dosage of biotin is up to 100 mcg, up to 96 mcg, up to 90 mcg, up 86 mcg, up to 80 mcg, up to 75 mcg, up to 70 mcg, up to 65 mcg, up to 60 mcg, up to 55 mcg, up to 50 mcg, up to 45 mcg, up to 40 mcg, up to 35 mcg, up to 30 mcg, up to 25 mcg, up to 20 mcg, up to 15 mcg, up to 10 mcg, up to 5 mcg, or up to 1 mcg.

Suitable oral formulations include tablets, gummies, gels, chewable tablets, dissolvable tablets, dissolvable sheets, microencapsulated capsules, elixirs, syrups, sachets, liquids, mouthwashes, tinctures or pastes. In certain embodiments, the composition is formulated for sublingual absorption. In certain embodiments, the composition is formulated for oral administration. Oral administration can allow for the absorption of the bioactive components of the composition via the mucous membranes of the mouth or via the intestinal tract. In some embodiments, the composition is administered orally as a gel. In some embodiments, the composition is administered as a dissolvable tablet that dissolves in the mouth. In some embodiments, the composition is administered as a gel-like sheet that dissolves on the tongue. In some embodiments, the composition is administered as a liquid, such as a mouthwash, ready-to-drink beverage, or a powder that can be dissolved in a liquid, such as water. In some embodiments, the composition is administered as a tincture. In certain embodiments, the composition is formulated as a dispersible powder, a beverage, a hard capsule, or a soft capsule. According to one or more embodiments, the composition is formulated for extended release, controlled release, or a combination thereof. In some embodiments, the composition is formulated for sustained or prolonged release. In certain embodiments, the composition comprises a pharmaceutically acceptable excipient.

Suitable topical formulations include gels, creams, lotions, ointments, salves, balms, and the like. The topical formulation may be formulation as an emulsion, foam, or solution. In some embodiments, the formulation is a solution. In some embodiments, the formulation is embedded, dispersed, coated, or deposited onto or in an adhesive patch. In some embodiments, the formulation is embedded, dispersed, coated, or deposited onto or in a solid “stick,” (that can be rubbed or sprayed onto the skin).

In some embodiments, the formulation is a hair care product. In some embodiments, the formulation is a shampoo. In some embodiments, the formulation is a conditioner. In some embodiments, the formulation is a leave-in conditioner. In some embodiments, the formulation is a mousse. In some embodiments, the formulation is a pomade. In some embodiments, the formulation is a hair spray.

In some embodiments, the formulation contains from about 2% to about 8% of the spray dried composition, the freeze dried composition, the milled composition, or the admixture of the present disclosure (w/w), from about 2% to about 7% of the spray dried composition, the freeze dried composition, the milled composition, or the admixture of the present disclosure (w/w), from about 2% to about 6% of the spray dried composition, the freeze dried composition, the milled composition, or the admixture of the present disclosure (w/w), from about 2% to about 5% of the spray dried composition, the freeze dried composition, the milled composition, or the admixture of the present disclosure (w/w), from about 2% to about 4% of the spray dried composition, the freeze dried composition, the milled composition, or the admixture of the present disclosure (w/w), from about 3% to about 4% of the spray dried composition, the freeze dried composition, the milled composition, or the admixture of the present disclosure (w/w), or about 4% of the spray dried composition, the freeze dried composition, the milled composition, or the admixture of the present disclosure (w/w).

### Definitions

Unless otherwise defined herein, scientific and technical terms used in this application shall have the meanings that are commonly understood by those of ordinary skill in the art.

Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art. Chemistry terms used herein, unless otherwise defined herein, are used according to conventional usage in the art, as exemplified by “The McGraw-Hill Dictionary of Chemical Terms,” Parker S., Ed., McGraw-Hill, San Francisco, C.A. (1985).

“Silicate” as used herein refers to a compound comprising silicon and oxygen, and having a formula of [SiO^{(4-2x)-}₄₋ₓ], wherein 0≤x<2. Silicates may also be described by “Q units,” wherein:
Total Si—OH content—(Q⁰, Q¹, Q², and Q³ units)/(Q⁰, Q¹, Q², Q³, and Q⁴ units)
Q⁰=not linked to a second silicate group (e.g. orthosilicic acid)
Q¹=linked to one other silicate group
Q²=linked to two silicate groups
Q³=linked to three silicate groups
Q⁴=linked to four silicate groups (no Si—OH available).
   Silicates include orthosilicic acid, stabilized orthosilicic acids, orthosilicates, oligosilicates, chain silicates, pyro silicate, cyclic silicates, silicon dioxide, colloidal silicates, sheet silicates or tectosilicates silicates.

“Solubility” as used herein refers to the degree to which the compositions defined herein dissolves into water and the resultant solution is free of visual sedimentation and particulates. The solubility may be expressed as the amount of the composition that will dissolve in a specified volume of water under particular conditions. For example, the solubility of the compositions disclosed herein may be expressed as the weight percent of the composition in water at a specified temperature. Solubility may be determined by mixing a defined weight of the composition in a defined mass of water for a period of time (e.g. 5 minutes), and observing the resultant solution for sediment and particulates. Solubility measurements are art-recognized as representing a minimum solubility—but those of ordinary skill in the art will recognize that materials may also be soluble at higher concentrations than a stated solubility. For example, “the solubility of the composition in water is at least 10 wt %” and grammatical variants mean that that the composition is soluble at 10 wt %, and may also be soluble at higher concentrations, such as 20%, 30%, 40%, 50%, etc. The maximum solubility of any composition, if of interest, may be determined without undue experimentation by those of ordinary skill in the art.

### Combinations

In some embodiments, the spray dried compositions, compositions, milled compositions, and admixtures disclosed herein further comprise one or more ingredients selected from: nitrate or a salt thereof, beetroot, beetroot extract, delta-tocotrienol, gamma-tocotrienol, geranylgeraniol, biotin, magnesium biotinate, chromium picolinate, chromium chloride, chromium histidinate, zinc picolinate, plant extracts containing 6-MBOA (6-methoxybenzoxazolinone), cetyl octanoate, cetyl nonanoate, cetyl decanoate, cetyl undecanoate, cetyl laurate, cetyl tridecanoate, cetyl myristate, cetyl pentadecanoate, cetyl palmitate, cetyl heptadecanoate, cetyl stearate, agmatine nitrate, agmatine nitrite, alanine-hydroxyproline nitrate, alanine-hydroxyproline nitrite, arginine nitrate, arginine nitrite, arginine peptide nitrate, arginine peptide nitrite, beetroot nitrate, beetroot nitrite, beta-alanine nitrate, beta-alanine nitrite, betaine nitrate, betaine nitrite, casein nitrate, casein nitrite, casein hydrolysate nitrate, casein hydrolysate nitrite, citrulline nitrate, citrulline nitrite, collagen nitrate, collagen nitrite, collagen hydrolysate nitrate, collagen hydrolysate nitrite, collagen peptide nitrate, collagen peptide nitrite, creatine nitrate, creatine nitrite, creatine peptide nitrate, creatine peptide nitrite, fava bean peptide nitrate, fava bean peptide nitrite, glycine nitrate, glycine nitrite, glycine-proline nitrate, glycine-proline nitrite, glycine-proline-hydroxyproline nitrate, glycine-proline-hydroxyproline nitrite, glutamine nitrate, glutamine nitrite, glutamine peptide nitrate, glutamine peptide nitrite, hydroxyproline-glycine nitrate, hydroxyproline-glycine nitrite, ile-pro-pro nitrate, ile-pro-pro nitrite, isoleucine nitrate, isoleucine nitrite, l-histidine nitrate, l-histidine nitrite, leu-lys-pro-val-pro nitrate, leu-lys-pro-val-pro nitrite, leucine nitrate, leucine nitrite, leucine peptide nitrate, leucine peptide nitrite, norvaline nitrate, norvaline nitrite, ornithine nitrate, ornithine nitrite, pea nitrate, pea nitrite, pea hydrolysate nitrate, pea hydrolysate nitrite, pea oligopeptide nitrate, pea oligopeptide nitrite, peptide-bonded arginine nitrate, peptide-bonded arginine nitrite, peptide-bonded bcaa nitrate, peptide-bonded bcaa nitrite, peptide-bonded creatine nitrate, peptide-bonded creatine nitrite, proline-hydroxyproline nitrate, proline-hydroxyproline nitrite, rice nitrate, rice nitrite, rice hydrolysate nitrate, rice hydrolysate nitrite, rice peptide (tyr-pro-leu) nitrate, rice peptide (tyr-pro-leu) nitrite, rice peptide (val-tyr) nitrate, rice peptide (val-tyr) nitrite, soy nitrate, soy nitrite, soy hydrolysate nitrate, soy hydrolysate nitrite, soy peptide nitrate, soy peptide nitrite, val-pro-pro nitrate, val-pro-pro nitrite, valine nitrate, valine nitrite, whey nitrate, whey nitrite, whey hydrolysate nitrate, and whey hydrolysate nitrite. In certain preferred embodiments, the spray dried compositions, compositions, milled compositions, and admixtures disclosed herein further comprise nitrate or a salt thereof, most preferably beetroot or beetroot extract. In further preferred embodiments, the beetroot extract is a beetroot powder comprising betacyanins (betanin, isobetanin, neobetanin, betanidin), betaxanthins (vulgaxanthin I and II, indicaxanthin), phenolic acids (ferulic, caffeic, p-coumaric, syringic, vanillic, and gallic acids), flavonoids (rutin, catechin, epicatechin, kaempferol, and quercetin derivatives), saponins, betaine (trimethylglycine), amino acids (proline, alanine, glutamine, arginine, and aspartic acid), vitamins (C, B6, and folate), minerals (potassium, magnesium, iron, zinc, manganese, and copper), organic acids (malic, citric, oxalic, succinic, and gluconic acids), sugars (sucrose, glucose, and fructose), and minor constituents such as tyrosine derivatives, phenethylamines, amides (like N-trans-feruloyltyramine), and phytosterols (β-sitosterol and campesterol).

### EXAMPLES

The invention now being generally described, it will be more readily understood by reference to the following examples, which are included merely for purposes of illustration of certain aspects and embodiments of the present invention, and are not intended to limit the invention.

### Example 1: Preparation of Spray Dried Composition 1

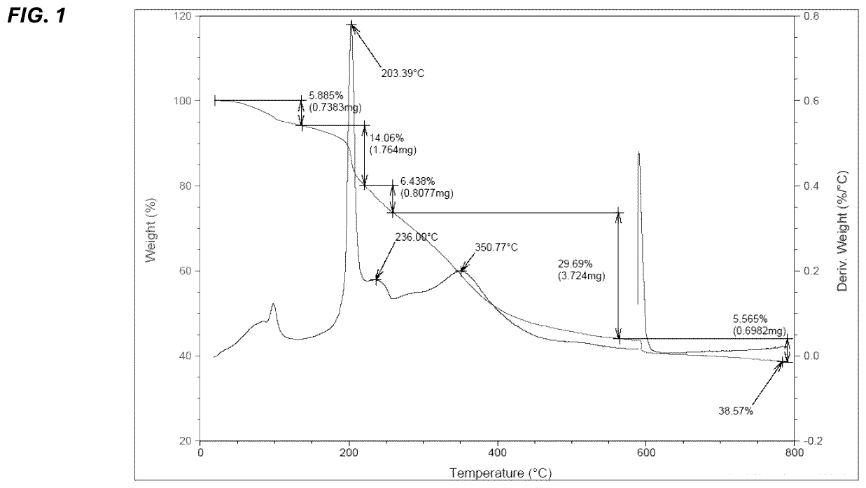

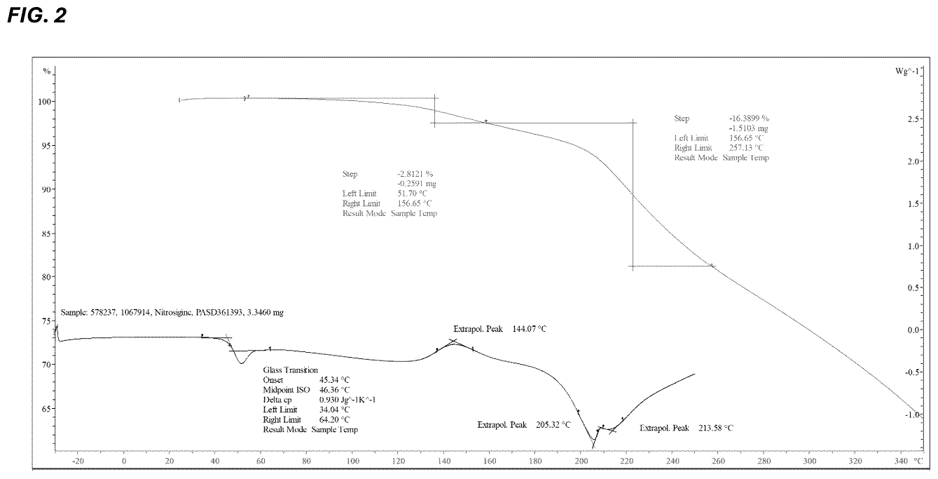

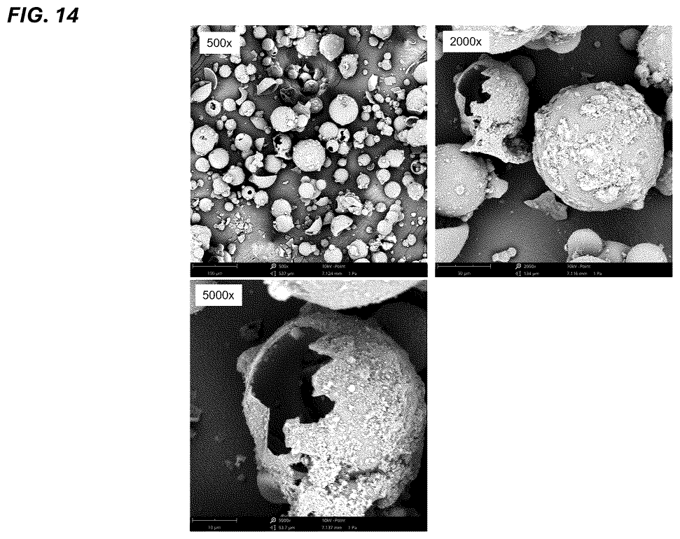

Water (40 g) was charged to a solution of potassium silicate (as 8.3% K₂O and 28.9% SiO₂) (84.0 g, 287.0 mmol SiO₂, 1 eq SiO₂,) and heated to 60° C. until completely dissolved. The reaction mixture was charged with inositol (25.6 g, 143.5 mmol, 0.5 eq) and maintained at 60° C. for ca. 20 min until completely dissolved. The reaction mixture was charged with L-arginine (50.0 g, 287.0 mmol, 1 eq) maintained at 60° C. for ca. 30 min until completely dissolved. The resultant solution was spray dried using with an inlet of 220° C. and an outlet temperature of 80° C. to afford Composition 1. FIG. 1 is a Thermogravimetric Analysis (TGA) thermogram of Composition 1.FIG. 2 is a Differential Scanning Calorimetry (DSC) thermogram of Composition 1. The DSC of Composition 1 exhibits a glass transition with an onset at 45.34° C., followed by an exothermic recrystallization at 144.07° C. Shortly thereafter the material melts at 205.32° C. and then decomposes. The TGA shows an overall weight loss of 19.202% before finally decomposing. FIG. 14 depicts SEM images of Composition 1.

### Example 2: Preparation of Composition 2

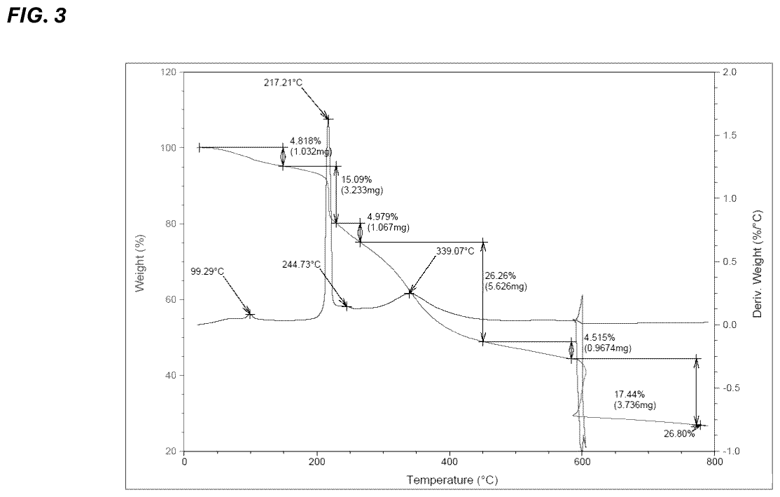

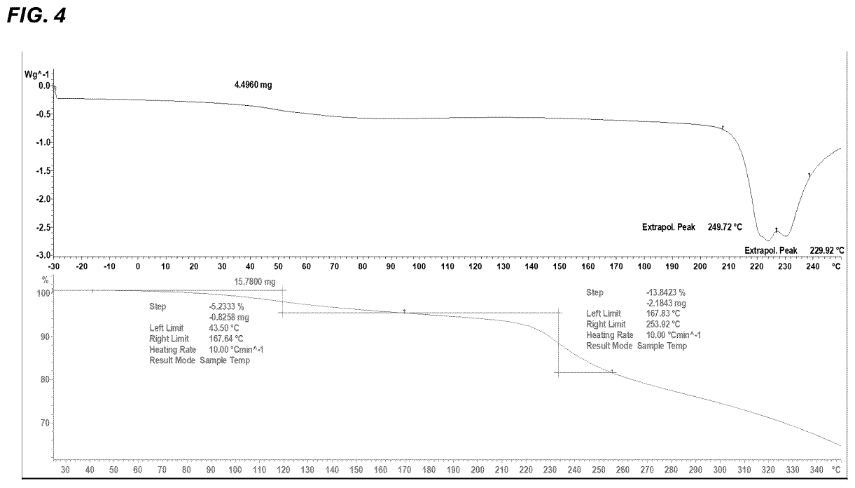

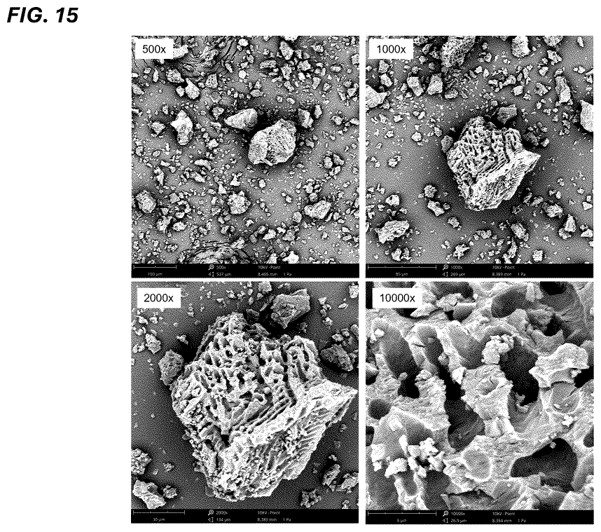

Water (8.06 g) was charged to a solution of potassium silicate (as 8.3% K₂O and 28.9% SiO₂) (16.8 g, 57.4 mmol SiO₂, 1 eq SiO₂) and heated to 60° C. until completely dissolved. The reaction mixture was charged with inositol (5.2 g, 28.7 mmol, 0.5 eq) and maintained at 60° C. for ca. 20 min until completely dissolved. The reaction mixture was charged with L-arginine (10.0 g, 57.4 mmol, 1 eq) maintained at 60° C. for ca. 30 min until completely dissolved. The solution was frozen with liquid nitrogen and freeze dried to afford Composition 2. FIG. 3 is a TGA thermogram of Composition 2. FIG. 4 is a DSC thermogram of Composition 2. The DSC of Composition 2 exhibits a glass transition with an onset at 45.34° C., followed by an exothermic recrystallization at 144.07° C. Shortly thereafter the material melts at 205.32° C. and then decomposes. The TGA shows an overall weight loss of 19.202% before finally decomposing. FIG. 15 depicts SEM images of Composition 2.

### Example 3: Preparation of Composition 3

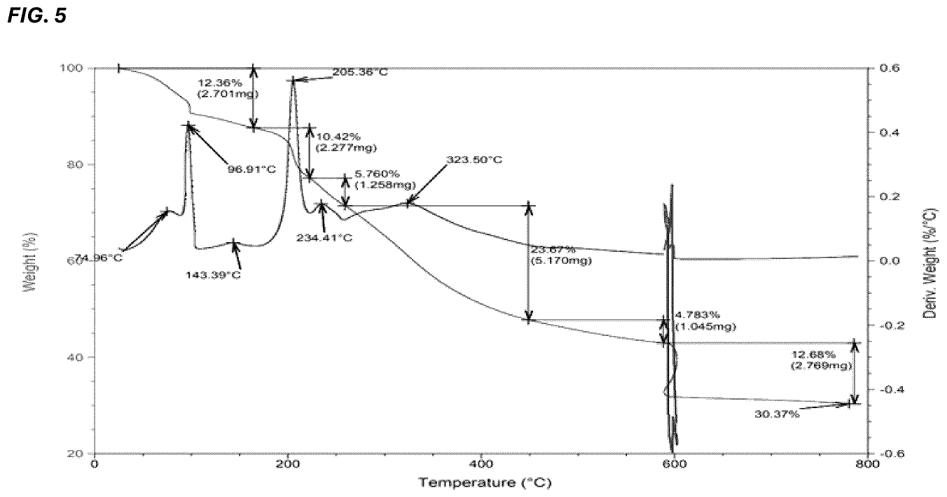

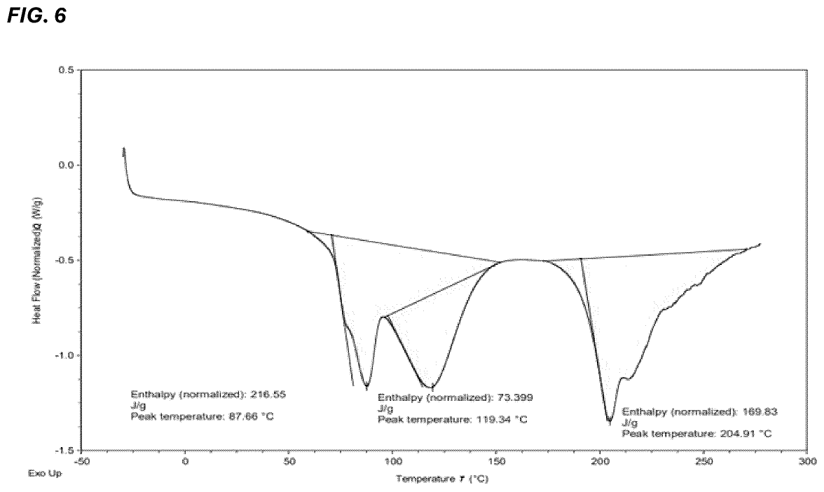

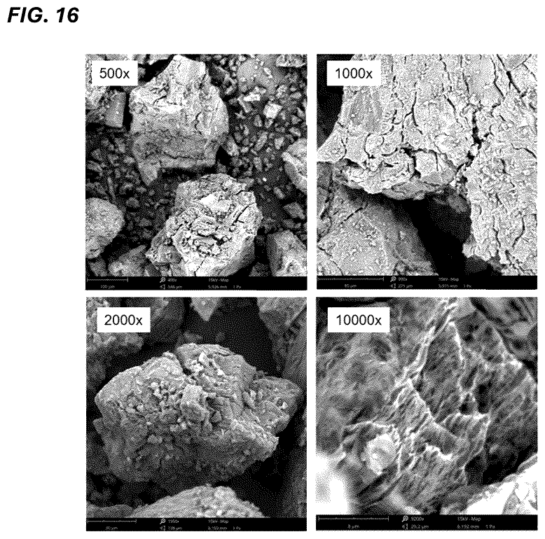

A solution of potassium silicate (as 8.3% K₂O and 28.9% SiO₂) (3.36 g, 11.5 mmol SiO₂, 1 eq SiO₂) and heated to 60° C. The solution was charged with inositol (1.0 g, 5.7 mmol, 0.5 eq) and maintained at 60° C. for ca. 20 min until completely dissolved. The reaction mixture was charged with L-arginine (2.0 g, 11.5 mmol, 1 eq) maintained at 60° C. for ca. 30 min until completely dissolved and then heated to 95° C. for 5 min to obtain a gel. The gel was transferred to a tray and dried overnight under vacuum at 75° C. for 4 days to afford Composition 3. FIG. 5 is a TGA thermogram of Composition 3. FIG. 6 is a DSC thermogram of Composition 3. FIG. 16 depicts SEM images of Composition 3.

### Example 4: Preparation of Spray Dried Composition 4

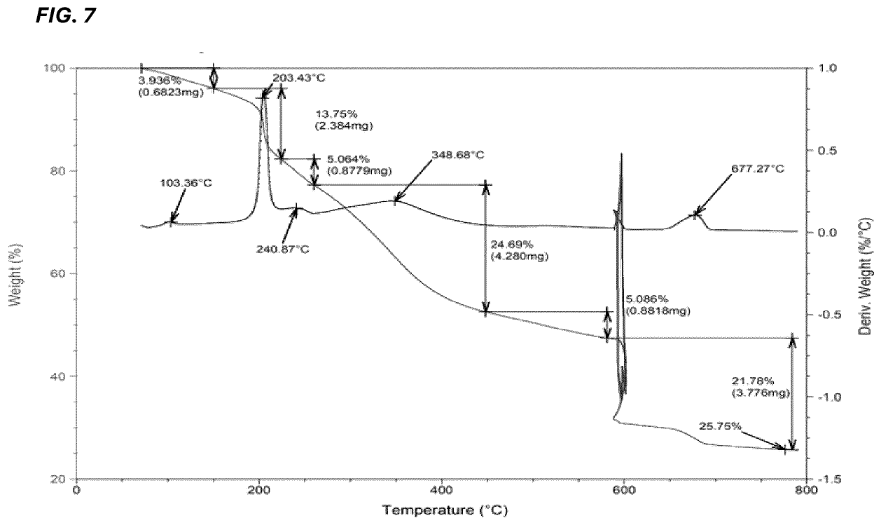

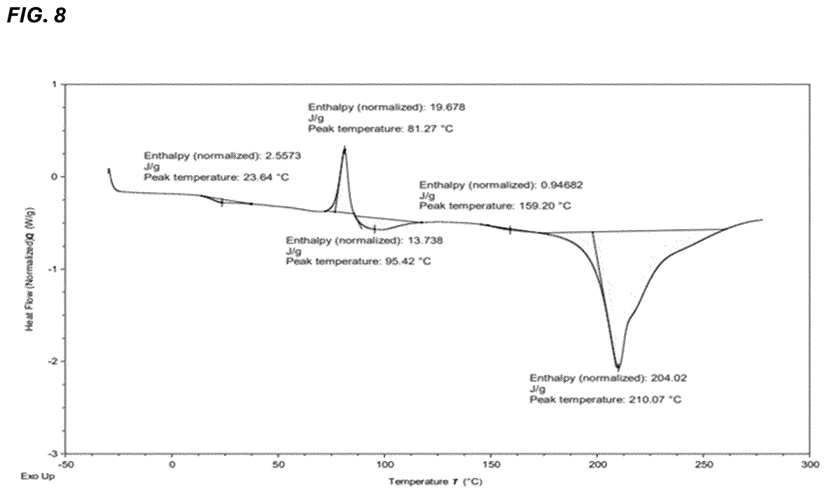

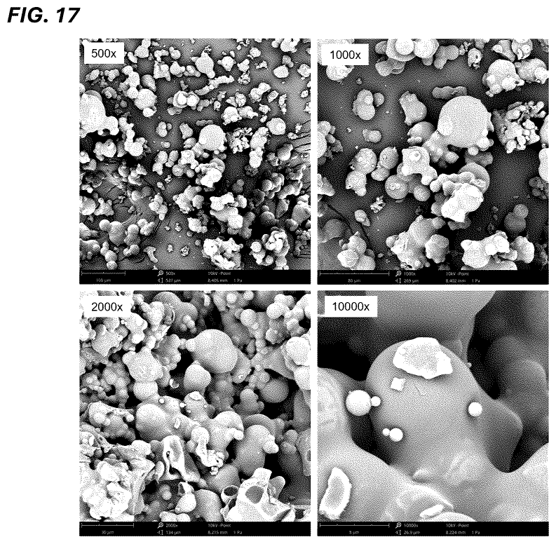

Water (40 g) was charged to a solution of potassium silicate (as 8.3% K₂O and 28.9% SiO₂) (84.0 g, 287.0 mmol SiO₂, 1 eq SiO₂,) and heated to 60° C. until completely dissolved. The reaction mixture was charged with inositol (25.6 g, 143.5 mmol, 0.5 eq) and maintained at 60° C. for ca. 20 min until completely dissolved. The reaction mixture was charged with L-arginine (50.0 g, 287.0 mmol, 1 eq) maintained at 60° C. for ca. 30 min until completely dissolved. A 45 wt % solution of potassium hydroxide (10 g, 80.4 mmol KOH, 0.3 eq) was added to the solution and stirred for 10 min. The resultant solution was spray dried with an inlet temperature of 220° C. and an outlet temperature of 80° C. to afford Composition 4. FIG. 7 is a TGA thermogram of Composition 4. FIG. 8 is a DSC thermogram of Composition 4. FIG. 17 depicts SEM images of Composition 4.

### Example 5: Preparation of Composition 5

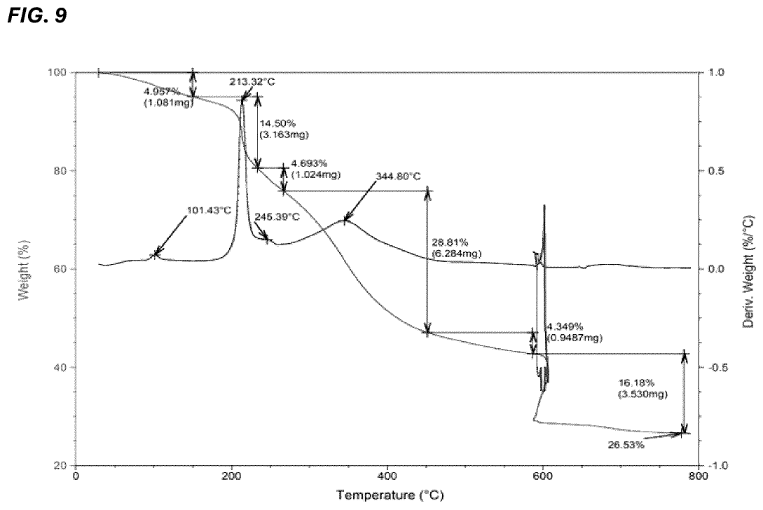

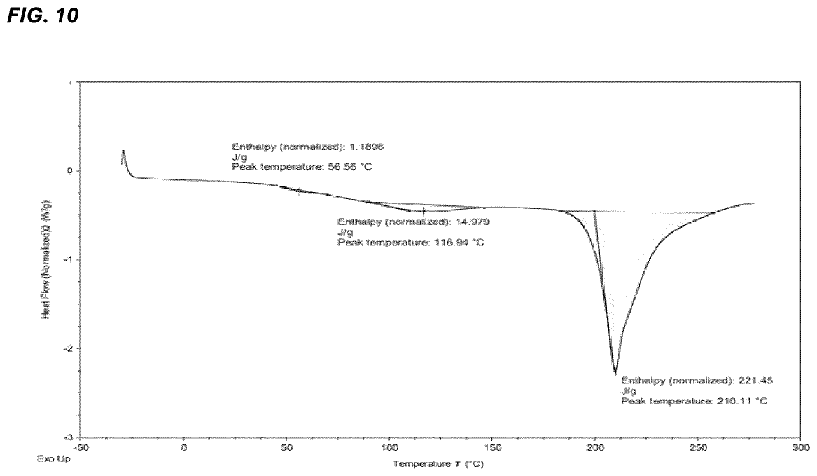

Water (20 g) was charged to a solution of potassium silicate (as 8.3% K₂O and 28.9% SiO₂) (42.0 g, 143.5 mmol SiO₂, 1 eq SiO₂) and heated to 60° C. until completely dissolved. The reaction mixture was charged with inositol (12.9 g, 71.8 mmol, 0.5 eq) and maintained at 60° C. for ca. 20 min until completely dissolved. The reaction mixture was charged with L-arginine (25.0 g, 143.5 mmol, 1 eq) maintained at 60° C. for ca. 30 min until completely dissolved. A 45 wt % solution of potassium hydroxide (5.2 g, 41.4 mmol KOH, 0.3 eq) was added to the solution and stirred for 10 min. The solution was frozen with liquid nitrogen and freeze dried to afford Composition 5. FIG. 9 is a TGA thermogram of Composition 5. FIG. 10 is a DSC thermogram of composition 5.

### Example 6: Preparation of Composition 6

Water (112 g) was charged to a solution of sodium silicate (as 8.3 wt % Na₂O and 28.9% SiO₂) (130.2 g, 574.1 mmol SiO₂, 1 eq SiO₂) and heated to 60° C. until completely dissolved. The reaction mixture was charged with inositol (51.7 g, 287.0 mmol, 0.5 eq) and maintained at 60° C. for ca. 20 min until completely dissolved. The reaction mixture was charged with L-arginine (100.0 g, 574.1 mmol, 1 eq) maintained at 60° C. for ca. 30 min until completely dissolved. A 50 wt % solution of sodium hydroxide (11.5 g, 143.5 mmol NaOH, 0.25 eq) was added to the solution and stirred for 10 min. The solution was frozen and freeze dried to afford Composition 6.

### Example 7: Preparation of Composition 7

Water (112 g) was charged to a solution of sodium silicate (as 8.3 wt % Na₂O and 28.9% SiO₂) (130.2 g, 574.1 mmol SiO₂, 1 eq SiO₂) and heated to 60° C. until completely dissolved. The reaction mixture was charged with inositol (51.7 g, 287.0 mmol, 0.5 eq) and maintained at 60° C. for ca. 20 min until completely dissolved. The reaction mixture was charged with L-arginine (100.0 g, 574.1 mmol, 1 eq) maintained at 60° C. for ca. 30 min until completely dissolved. A 50 wt % solution of sodium hydroxide (6.7 g, 143.5 mmol NaOH, 0.15 eq) was added to the solution and stirred for 10 min. The solution was frozen and freeze dried to afford Composition 7.

### Example 8: Preparation of Composition 8

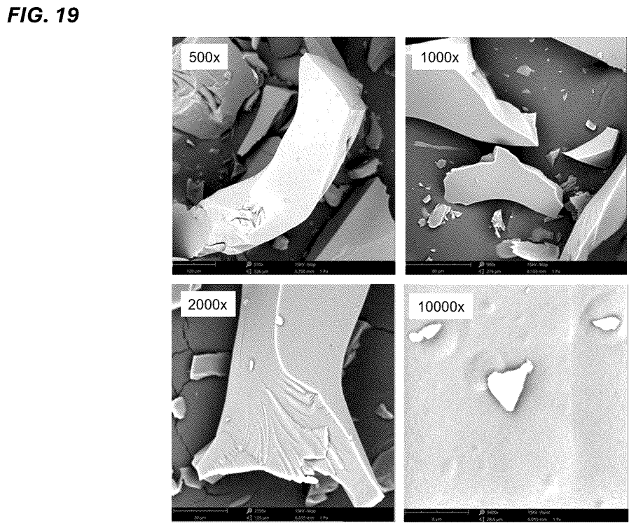

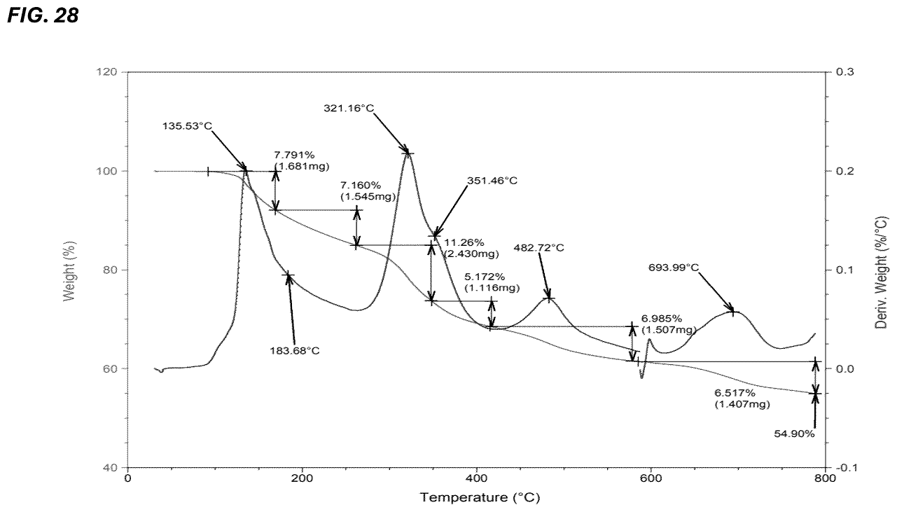

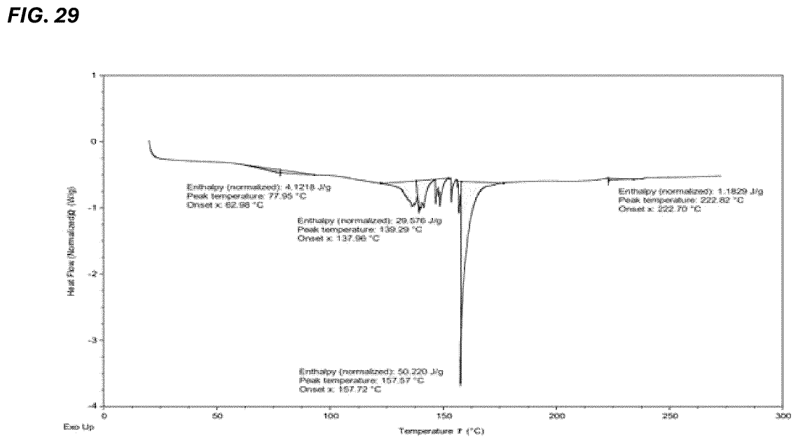

A solution of sodium silicate (as 8.3 wt % Na₂O and 28.9% SiO₂) (130.2 g, 574.1 mmol SiO₂, 1 eq SiO₂) was heated to 60° C. The solution was charged with inositol (51.7 g, 287.0 mmol, 0.5 eq) and maintained at 60° C. for ca. 20 min until completely dissolved. A 50 wt % solution of sodium hydroxide (13.8 g, 172.2 mmol NaOH, 0.3 eq) was added to the solution and stirred for 10 min. The solution was frozen and freeze dried to afford Composition 8. FIG. 19 depicts SEM images of Composition 8. FIG. 28 is a TGA thermogram of Composition 8. FIG. 29 is a DSC thermogram of Composition 8; this material shows a small transition occurring between 62° C. and 77° C. A major transition occurs between 137° C. and 160° C.

### Example 9: Preparation of Composition 9

Water (60 mL) was charged with NaOH (1.5 g) and stirred to afford a clear solution. The solution was charged Composition 1 (30 g) and heated to 80° C. for 5 min until the solids were completely dissolved and then allowed to cool to rt to afford Composition 9 as a clear solution with no visible sedimentation. The material is completely soluble at 50 wt % in water.

### Example 10: Preparation of Composition 10

Water (20 g) was charged to a solution of potassium silicate (as 8.3% K₂O and 28.9% SiO₂) (42.0 g, 143.5 mmol SiO₂, 1 eq SiO₂) and heated to 60° C. until completely dissolved. The reaction mixture was charged with inositol (12.9 g, 71.8 mmol, 0.5 eq) and maintained at 60° C. for ca. 20 min until completely dissolved. The reaction mixture was charged with L-arginine (25.0 g, 143.5 mmol, 1 eq) maintained at 60° C. for ca. 30 min until completely dissolved. A 45 wt % solution of potassium hydroxide (5.4 g, 43.1 mmol KOH, 0.3 eq) was added to the solution and stirred for 10 min and allowed to cool to rt to afford Composition 10 as a clear solution (50 wt %) with no visible sedimentation.

### Example 11: Preparation of Composition 11

Water (125 g) was charged to a solution of potassium silicate (as 8.3% K₂O and 28.9% SiO₂) (42.0 g, 143.5 mmol SiO₂, 1 eq SiO₂) and heated to 60° C. until completely dissolved. The reaction mixture was charged with inositol (12.9 g, 71.8 mmol, 0.5 eq) and maintained at 60° C. for ca. 20 min until completely dissolved. The reaction mixture was charged with L-arginine (25.0 g, 143.5 mmol, 1 eq) maintained at 60° C. for ca. 30 min until completely dissolved. A 45 wt % solution of potassium hydroxide (5.4 g, 43.1 mmol KOH, 0.3 eq) was added to the solution and stirred for 10 min and allowed to cool to rt to afford Composition 11 as a clear solution (25 wt %) with no visible sedimentation.

### Example 12: Preparation of Composition 12

A solution of potassium silicate (as 8.3% K₂O and 28.9% SiO₂) (8.4 g, 28.7 mmol SiO₂, 1 eq SiO₂) and heated to 60° C. The reaction mixture was charged with inositol (2.6 g, 14.4 mmol, 0.5 eq) and maintained at 60° C. for ca. 20 min until completely dissolved. The solution was transferred to a tray and dried overnight under vacuum at 45° C. for 16 h to afford Composition 12.

### Example 13: Preparation of Composition 13

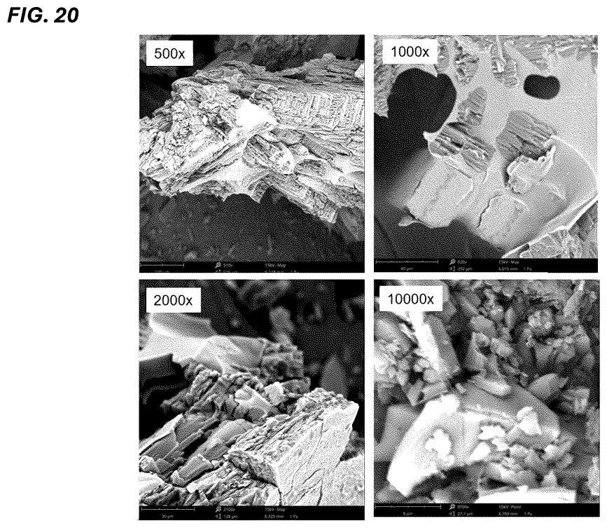

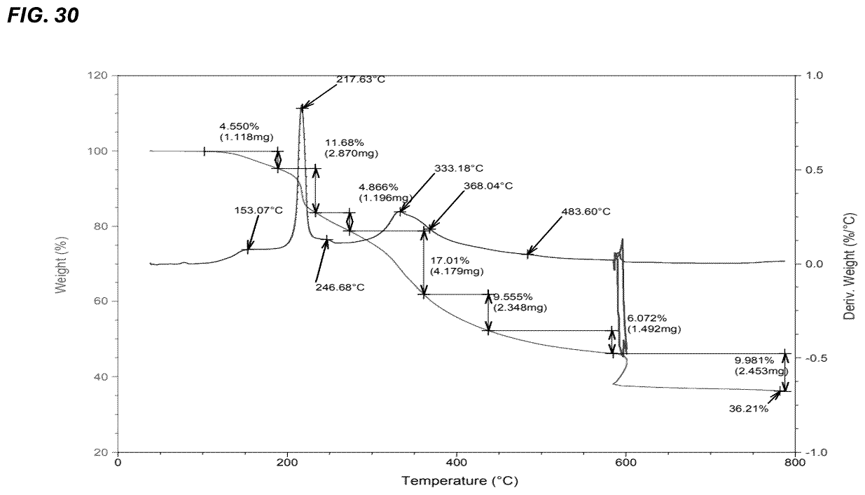

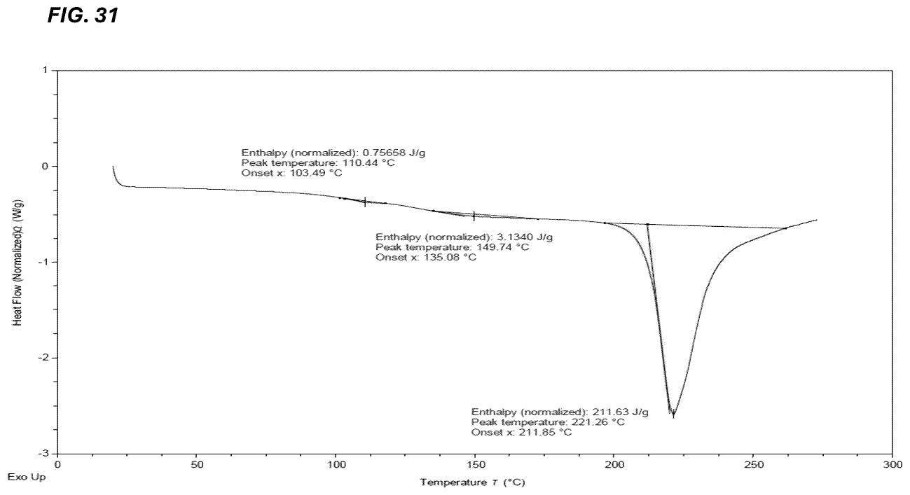

Water (224 g) was charged to a solution of sodium silicate (as 8.3 wt % Na₂O and 28.9% SiO₂) (260.3 g, 1148 mmol SiO₂, 1 eq SiO₂) and heated to 60° C. until completely dissolved. The reaction mixture was charged with inositol (103.4 g, 574 mmol, 0.5 eq) and maintained at 60° C. for ca. 20 min until completely dissolved. The reaction mixture was charged with L-arginine (200.0 g, 1148 mmol, 1 eq) maintained at 60° C. for ca. 30 min until completely dissolved. The solution was frozen and freeze dried to afford Composition 13. FIG. 20 depicts SEM images of Composition 13. FIG. 23 is an XRPD pattern of Composition 13. FIG. 30 is a TGA thermogram of Composition 13. FIG. 31 is a DSC thermogram of Composition 13; this material shows minor transitions until temperatures above 210° C. are reached.

### Example 14: Preparation of Composition 14

Water (337 g) was charged to a solution of sodium silicate (as 9.1 wt % Na₂O and 29.0% SiO₂) (237.86 g, 1148 mmol SiO₂, 1 eq SiO₂) and heated to 60° C. until completely dissolved. The reaction mixture was charged with inositol (103.42 g, 574.1 mmol, 0.5 eq) and maintained at 60° C. for ca. 20 min until completely dissolved. The reaction mixture was charged with L-arginine (200.0 g, 1148 mmol, 1 eq) and heated to 75° C. for ca. 30 min until completely dissolved. The solution was frozen and freeze dried with the following parameters to afford Composition 14: Drying temperature (200° F.); Pressure (3000-4000 mtor); Drying Time (21 h).

### Example 15: Preparation of Composition 15

Water (337 g) was charged to a solution of sodium silicate (as 9.1 wt % Na₂O and 29.0% SiO₂) (237.86 g, 1148 mmol SiO₂, 1 eq SiO₂) and heated to 60° C. until completely dissolved. The reaction mixture was charged with inositol (103.42 g, 574.1 mmol, 0.5 eq) and maintained at 60° C. for ca. 20 min until completely dissolved. The reaction mixture was charged with L-arginine (200.0 g, 1148 mmol, 1 eq) and heated to 75° C. for ca. 30 min until completely dissolved. The solution was frozen and freeze dried with the following parameters to afford Composition 15: Drying temperature (180° F.); Pressure (100-150 mtor); Drying Time (25 h).

### Example 16: Preparation of Composition 16

Water (337 g) was charged to a solution of sodium silicate (as 9.1 wt % Na₂O and 29.0% SiO₂) (237.86 g, 1148 mmol SiO₂, 1 eq SiO₂) and heated to 60° C. until completely dissolved. The reaction mixture was charged with inositol (103.42 g, 574.1 mmol, 0.5 eq) and maintained at 60° C. for ca. 20 min until completely dissolved. The reaction mixture was charged with L-arginine (200.0 g, 1148 mmol, 1 eq) and heated to 75° C. for ca. 30 min until completely dissolved. A 50 wt % solution of sodium hydroxide (23.0 g, 287 mmol NaOH, 0.25 eq) was added to the solution and stirred for 10 min. The solution was frozen and freeze dried with the following parameters to afford Composition 16: Drying temperature (200° F.); Pressure (3000-4000 mtor); Drying Time (21 h).

### Example 17: Preparation of Composition 17

Water (337 g) was charged to a solution of sodium silicate (as 9.1 wt % Na₂O and 29.0% SiO₂) (237.86 g, 1148 mmol SiO₂, 1 eq SiO₂) and heated to 60° C. until completely dissolved. The reaction mixture was charged with inositol (103.42 g, 574.1 mmol, 0.5 eq) and maintained at 60° C. for ca. 20 min until completely dissolved. The reaction mixture was charged with L-arginine (200.0 g, 1148 mmol, 1 eq) and heated to 75° C. for ca. 30 min until completely dissolved. A 50 wt % solution of sodium hydroxide (23.0 g, 287 mmol NaOH, 0.25 eq) was added to the solution and stirred for 10 min. The solution was frozen and freeze dried with the following parameters to afford Composition 17: Drying temperature (180° F.); Pressure (100-150 mtor); Drying Time (25 h).

### Example 18: Preparation of Composition 18

Water (337 g) was charged to a solution of sodium silicate (as 9.1 wt % Na₂O and 29.0% SiO₂) (237.86 g, 1148 mmol SiO₂, 1 eq SiO₂) and heated to 60° C. until completely dissolved. The reaction mixture was charged with inositol (103.42 g, 574.1 mmol, 0.5 eq) and maintained at 60° C. for ca. 20 min until completely dissolved. The reaction mixture was charged with L-arginine (200.0 g, 1148 mmol, 1 eq) and heated to 90° C. for ca. 30 min until completely dissolved. The solution was frozen and freeze dried with the following parameters to afford Composition 18: Drying temperature (200° F.); Pressure (3000-4000 mtor); Drying Time (21 h).

### Example 19: Preparation of Composition 19

Water (337 g) was charged to a solution of sodium silicate (as 9.1 wt % Na₂O and 29.0% SiO₂) (237.86 g, 1148 mmol SiO₂, 1 eq SiO₂) and heated to 60° C. until completely dissolved. The reaction mixture was charged with inositol (103.42 g, 574.1 mmol, 0.5 eq) and maintained at 60° C. for ca. 20 min until completely dissolved. The reaction mixture was charged with L-arginine (200.0 g, 1148 mmol, 1 eq) and heated to 90° C. for ca. 30 min until completely dissolved. The solution was frozen and freeze dried with the following parameters to afford Composition 19: Drying temperature (180° F.); Pressure (100-150 mtor); Drying Time (25 h).

### Example 20: Preparation of Composition 20

Water (337 g) was charged to a solution of sodium silicate (as 9.1 wt % Na₂O and 29.0% SiO₂) (237.86 g, 1148 mmol SiO₂, 1 eq SiO₂) and heated to 60° C. until completely dissolved. The reaction mixture was charged with inositol (103.42 g, 574.1 mmol, 0.5 eq) and maintained at 60° C. for ca. 20 min until completely dissolved. The reaction mixture was charged with L-arginine (200.0 g, 1148 mmol, 1 eq) and heated to 90° C. for ca. 30 min until completely dissolved. A 50 wt % solution of sodium hydroxide (23.0 g, 287 mmol NaOH, 0.25 eq) was added to the solution and stirred for 10 min. The solution was frozen and freeze dried with the following parameters to afford Composition 20: Drying temperature (200° F.); Pressure (3000-4000 mtor); Drying Time (21 h).

### Example 21: Preparation of Composition 21

Water (337 g) was charged to a solution of sodium silicate (as 9.1 wt % Na₂O and 29.0% SiO₂) (237.86 g, 1148 mmol SiO₂, 1 eq SiO₂) and heated to 60° C. until completely dissolved. The reaction mixture was charged with inositol (103.42 g, 574.1 mmol, 0.5 eq) and maintained at 60° C. for ca. 20 min until completely dissolved. The reaction mixture was charged with L-arginine (200.0 g, 1148 mmol, 1 eq) and heated to 90° C. for ca. 30 min until completely dissolved. A 50 wt % solution of sodium hydroxide (23.0 g, 287 mmol NaOH, 0.25 eq) was added to the solution and stirred for 10 min. The solution was frozen and freeze dried with the following parameters to afford Composition 21: Drying temperature (180° F.); Pressure (100-150 mtor); Drying Time (25 h).

### Characterization Analysis of Compositions 20 and 21

Representative procedure to prepare Composition 21 NMR sample: 75.4 mg of Composition 21 was dissolved in approximately 1.0 mL of D₂O.

Representative procedure to prepare Composition 20 NMR sample: 87.7 mg of Composition 20 was dissolved in approximately 1.0 mL of D₂O.

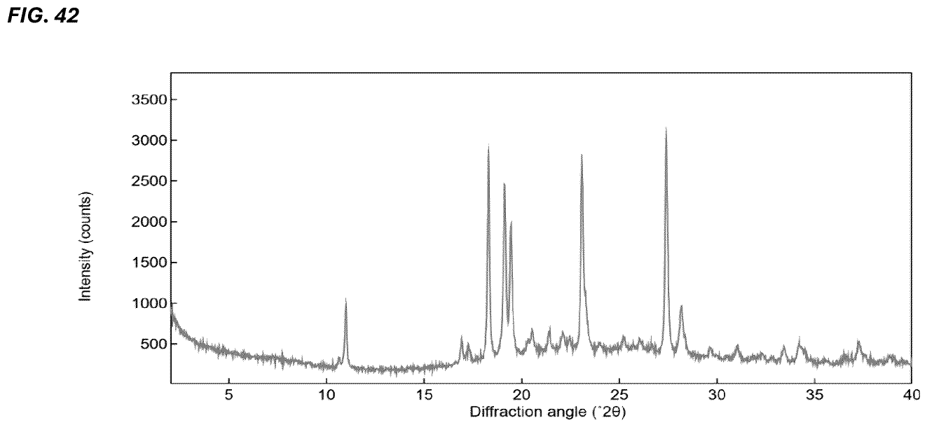

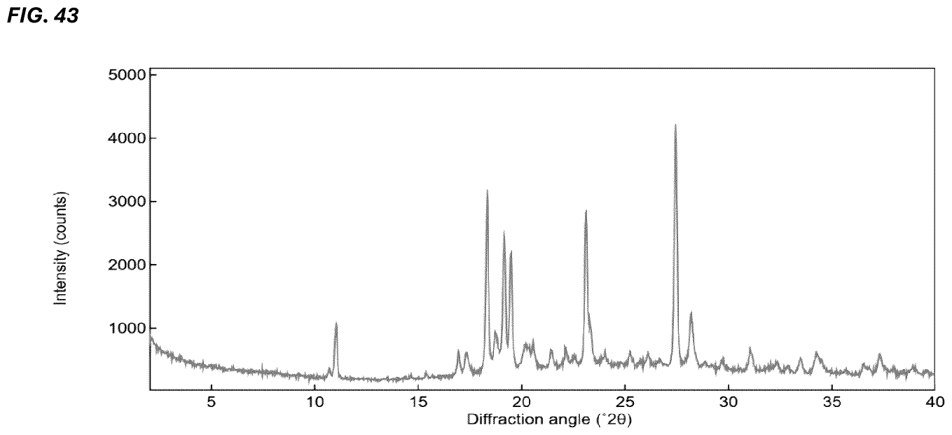

XRPD analyses of Compositions 20 and 21 revealed that each sample displayed sharp, distinct peaks (FIG. 42 and FIG. 43), indicating that the samples were composed of a crystalline form or a mixture of crystalline forms.

Comparative analysis of the two acquired XRPD patterns for the two compositions show slight differences. A peak at 20.2° 2θ displays a much lower intensity in Composition 21 than in Composition 20. This intensity difference between the two compositions is likely due to preferred orientation (PO). Alternatively, it is possible that there is a slight difference in concentration of the components between the two samples. PO refers to a phenomenon where certain crystallographic planes are aligned more frequently in a specific di-rection within a sample leading to artificial attenuation or enhancement of a peak intensity.

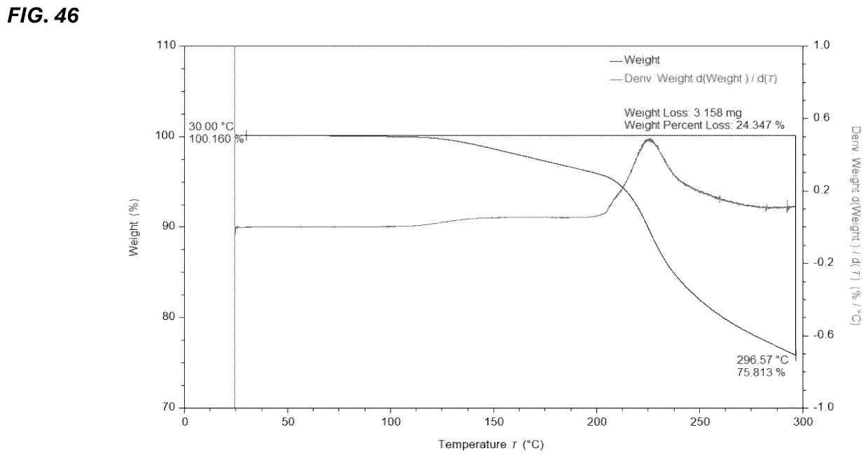

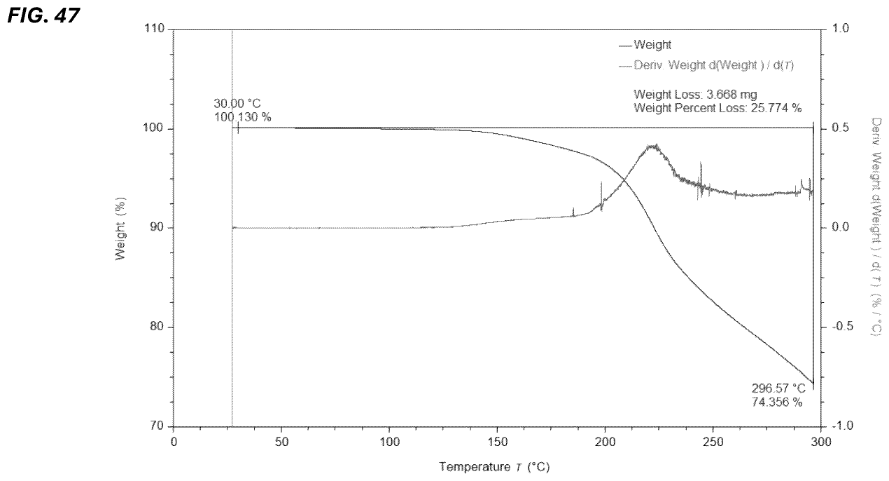

Compositions 20 and 21 were also analyzed by TGA. Each sample showed a similar profile with minor, gradual weight loss and a subsequent, significant step change which occurred at −200° C. The step-wise weight loss is most likely due to the decomposition of the materials based on the continual weight loss extending to higher temperatures (FIG. 46 and FIG. 47). The samples lost between 24 and 26% mass across the analyzed temperature range. Composition 21 resulted in 24.347% weight loss from 10 to 296.57° C., while Composition 20 resulted in 25.774% weight loss from 10 to 296.57° C.

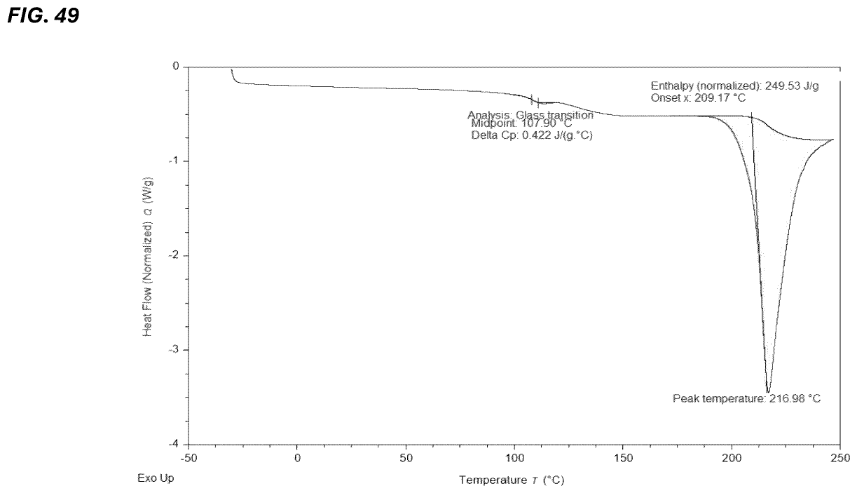

Compositions 20 and 21 were also analyzed by DSC. The resulting thermograms were significantly different. The thermogram of Composition 21 (FIG. 49) displayed a glass transition at 107.90° C. followed by an endothermic event with an onset of 209.17° C. The observation of the glass transition indicated that at least one component in Composition 21 transitioned from a rigid, glassy state to a more flexible state, a typical property of amorphous material. Inositol and L-arginine are materials that can display crystallinity with melting points of ˜225° C. and 223-244° C., respectively. Potassium silicate displays a melt at 905° C. Since the XRPD pattern for Composition 21 displayed crystallinity, it is suspected that the broad endotherm at 209.17° C. may be the melting of inositol, L-arginine, or both components.

It is inconclusive whether Composition 20 thermogram displayed a glass transition. The DSC for Composition 20 displayed a baseline offset at a higher temperature (˜125° C.) as compared to Composition 21 (107.90° C.) but was convoluted with the broader endothermic event at 212.08° C. Analogous to the Composition 21's DSC, the thermal transition at 212.08° C. is suspected to be attributed to inositol and/or L-arginine melting. The DSC results are summarized in Table 1.

TABLE 1
DSC results for Compositions 21 and 20

| Sample | Results |
|---|---|
| Composition 21 | Endotherm Apex: 216.98° C. |
| | Endotherm Onset: 209.17° C. |
| | Endotherm Enthalpy: 249.53 J/g |
| | Glass Transition: 107.90° C. |
| Composition 20 | Endotherm Apex: 212.08° C. |
| | Endotherm Onset: 194.02° C. |
| | Endotherm Enthalpy: 292.48 J/g |

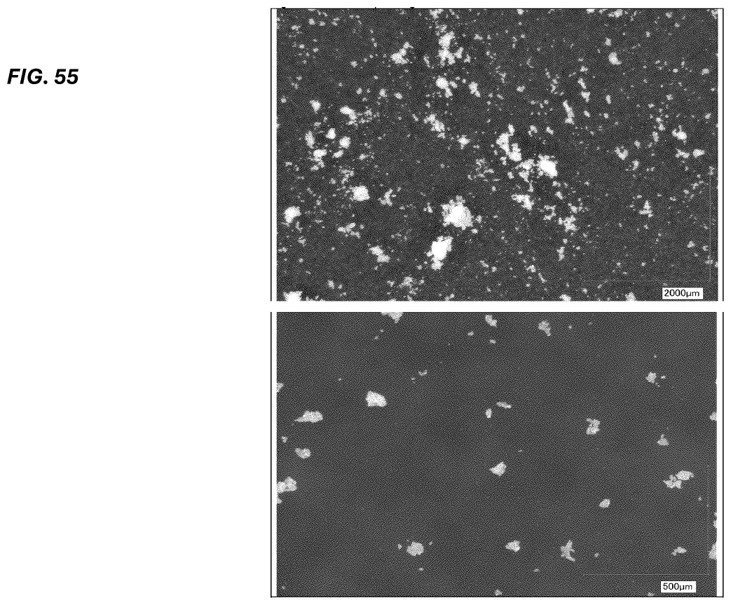

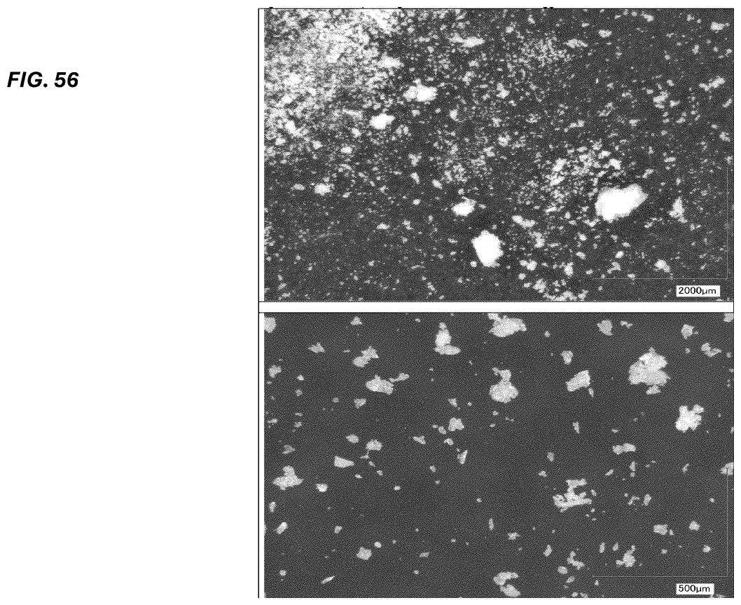

Using a digital microscope, optical images of Compositions 20 and 21 were also acquired (FIG. 55 and FIG. 56). Both compositions displayed conglomerates of plate-like particles and individual plate-like particles of various sizes and shapes.

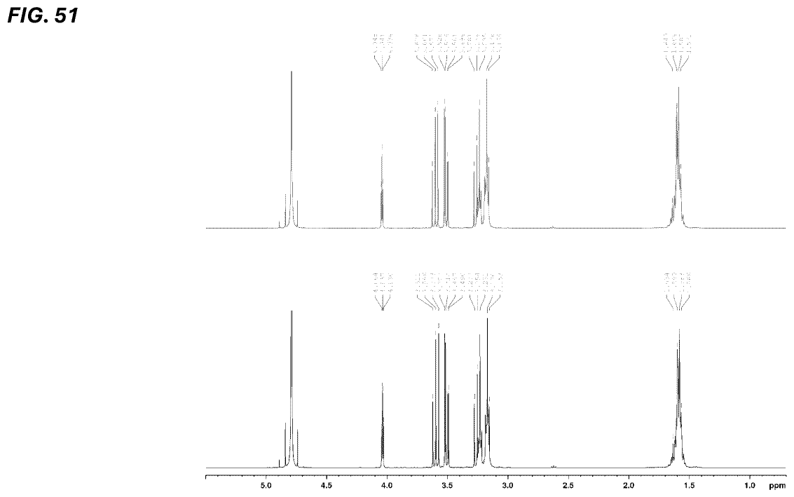

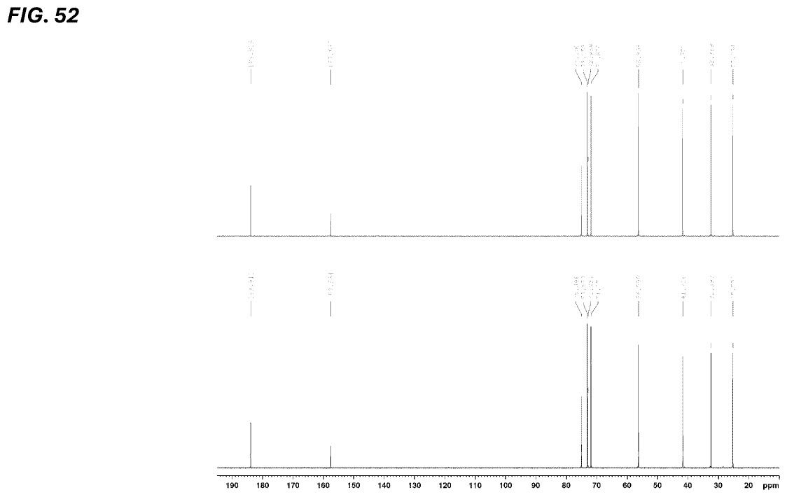

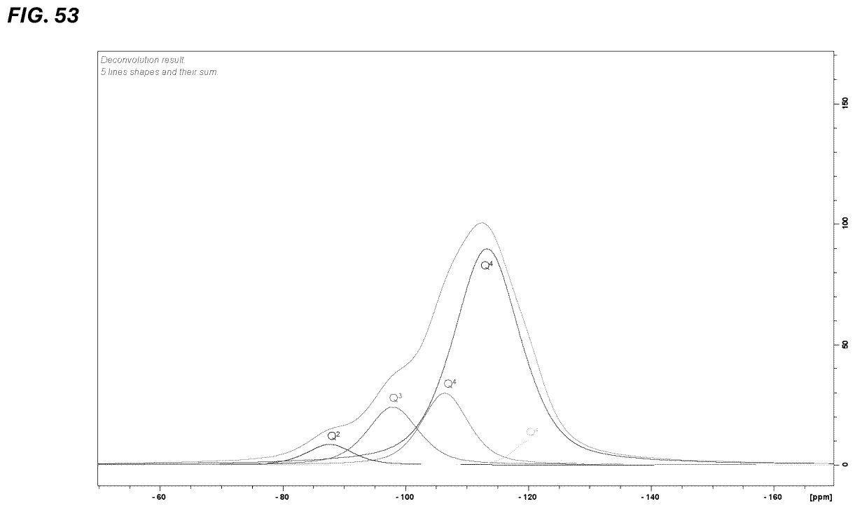

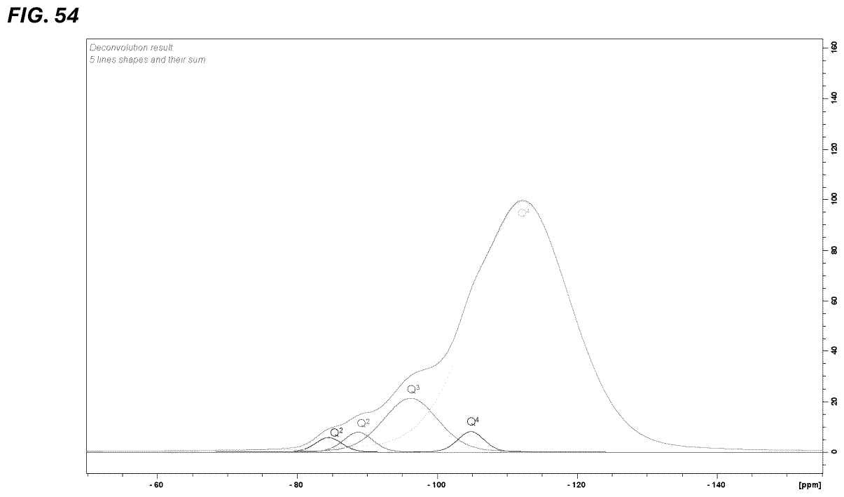

Compositions 20 and 21 were submitted for solution state ¹H (FIG. 51) and ¹³C (FIG. 52) NMR analyses, as well as ²⁹Si ssNMR (FIG. 53 and FIG. 54) to determine the Qⁿ distribution and the total Si—OH content in the samples. Based on the comparative analysis of the ¹H and ¹³C NMR spectra, the chemical shifts (peak positions) and multiplicity (coupling patterns) observed in both spectra are consistent, indicating that both samples contain the same components. However, the discrepancy in the ¹H integration values imply a variation in the relative quantities of these components between the two samples.

FIG. 53 and FIG. 54 display the ²⁹Si ssNMR spectra of the two samples. The chemical shifts, area intensity (integration), and the percentages of the Qⁿ units, including the total Si—OH content of each sample, are summarized in Tables 2 and 3. The values reported are rounded to the second decimal point after the calculations are completed. The NMR data demonstrated a discrepancy in the ¹H integration values implying a variation in the relative quantities of the individual components of spray dried Composition 22 (L-arginine, potassium silicate, inositol).

TABLE 2
²⁹Si ssNMR chemical shift and area intensity
(integration) for Qⁿ units

| | Chemical Shift | | | |
|---|---|---|---|---|
| Sample | (ppm) | Area | Rel. Area (%) | ^{Qn} unit |
| Composition 21 | −87.6 | 13225 | 4.24% | Q² |
| | −97.9 | 40962 | 13.12% | Q³ |
| | −106.4 | 46781 | 14.98% | Q⁴ |
| | −113.2 | 200248 | 64.14% | Q⁴ |
| | −120.3 | 10978 | 3.52% | Q⁴ |
| Composition 20 | −84.4 | 3948 | 1.38% | Q² |
| | −88.7 | 5964 | 2.08% | Q² |
| | −96.2 | 30028 | 10.47% | Q³ |
| | −104.8 | 5552 | 1.94% | Q⁴ |
| | −112.3 | 241320 | 84.14% | Q⁴ |

TABLE 3
% of the Qⁿ units and the total Si—OH content

| | | Q³ | Q⁴ | Total Si—OH |
|---|---|---|---|---|
| Sample | Q²Unit (%) | Unit (%) | Unit (%) | Content (%) |
| Composition 21 | 4.24% | 13.12% | 82.64% | 21.59% |
| Composition 20 | 3.46% | 10.47% | 86.07% | 17.38% |

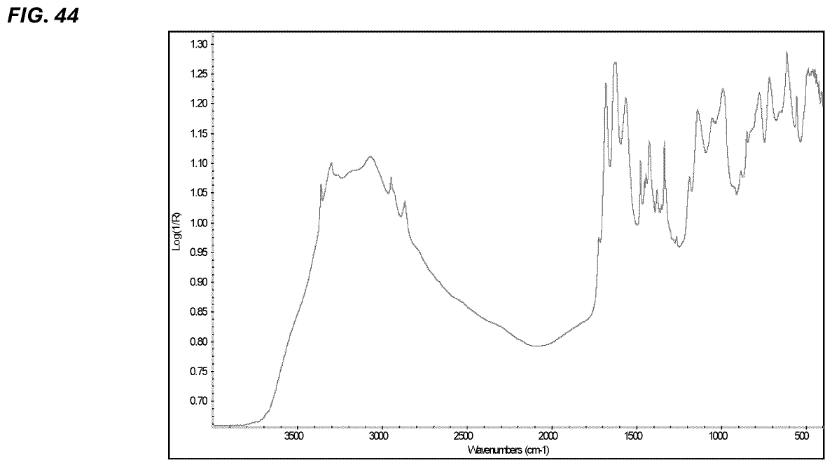

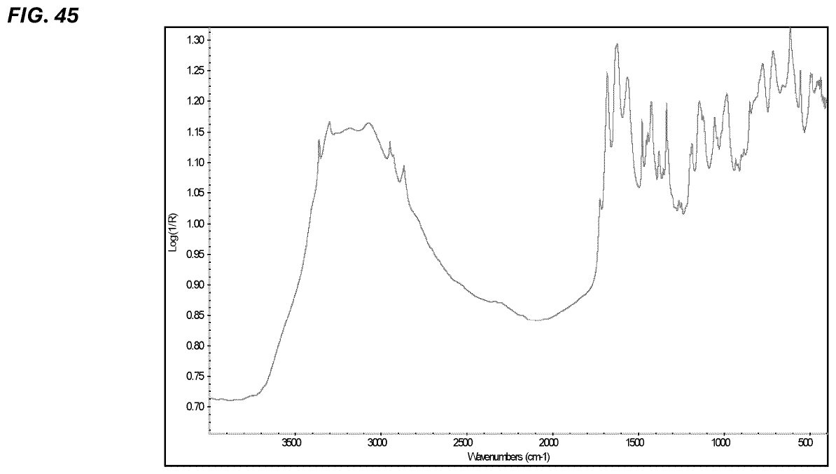

A comparison of the IR spectra showed slight differences in peak positions and relative intensities between the two samples (FIG. 44 and FIG. 45). Peaks at approximately 899, 892, and 1246 cm⁻¹ were absent in Composition 21 but present in Composition 20, indicating differences between the two materials.

### Example 22: Preparation of Spray Dried Composition 22

Spray dried Composition 22 is a physical mixture product combining L-arginine, potassium silicate, and inositol. A series of characterization techniques, including powder X-ray diffraction (XRPD), infrared (IR) spectroscopy, thermogravimetry analysis (TGA), differential scanning calorimetry (DSC), nuclear magnetic resonance (NMR), and optical microscopy (OM), were performed involving various amounts and sample preparation techniques.

Representative Procedure to prepare spray dried Composition 22 IR sample: Each sample was prepared by diluting approximately 50:1 by mass in KBr and grinding the mixture into a fine powder using a mortar and pestle. Each diluted sample was packed into a micro sample cup with a smooth, level surface. Signal acquisition was optimized using a sample accessory mirror and adjusting the stage height to maximize the peak-to-peak value.

Representative Procedure to prepare spray dried Composition 22 NMR sample: 96.0 mg of Composition 22 was added to approximately 1.0 mL of D₂O. The solution was sonicated, centrifuged, and filtered using a 0.45 μm nylon syringe filter. The subsequent supernatant was collected and analyzed by ¹H and ¹³C NMR spectroscopy.

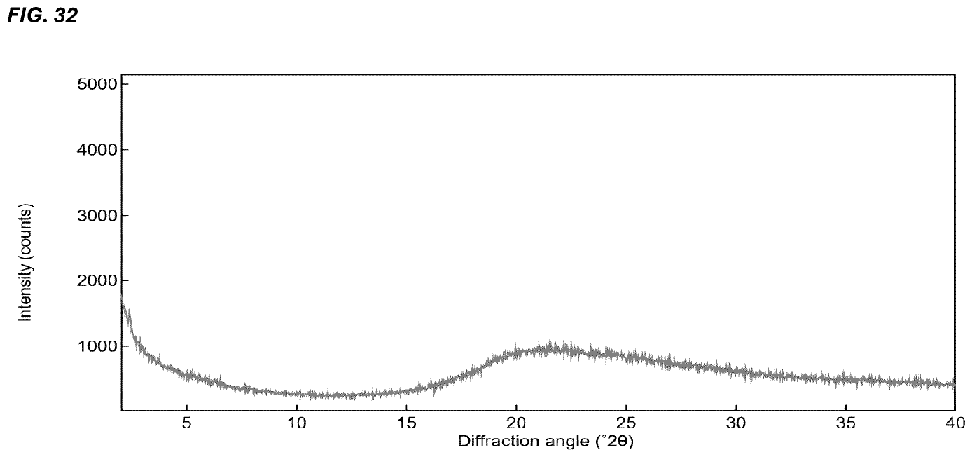

XRPD analysis of the spray dried composition revealed that the sample displayed a broad, diffuse diffraction response that lacked sharp, distinct peaks (FIG. 32), indicating that the sample was composed of an amorphous form or a mixture of amorphous forms. XRPD analysis revealed that spray dried Composition 22 is an amorphous solid.

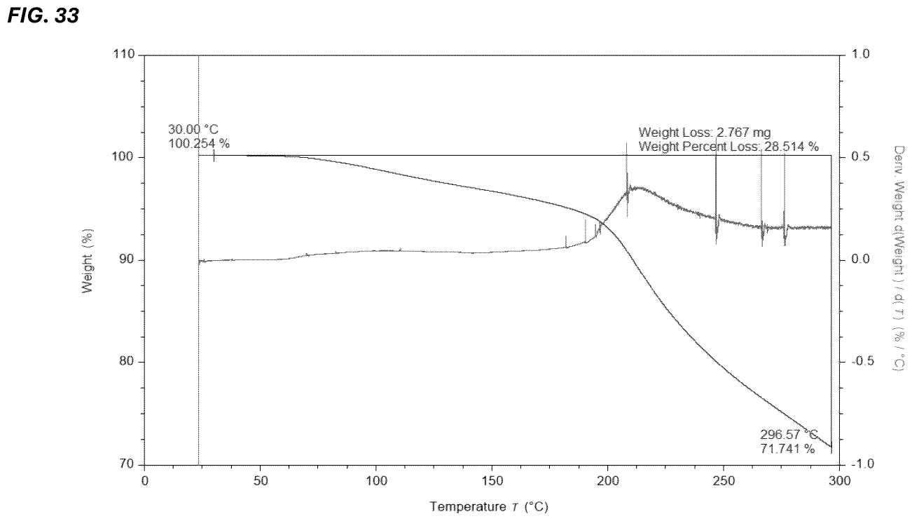

TGA analysis showed a minor, gradual weight loss until a significant step change occurred around 200° C., likely due to the decomposition of the materials based on the continual weight loss above that temperature (FIG. 33). The sample lost approximately 28.5% mass across the analyzed temperature range.

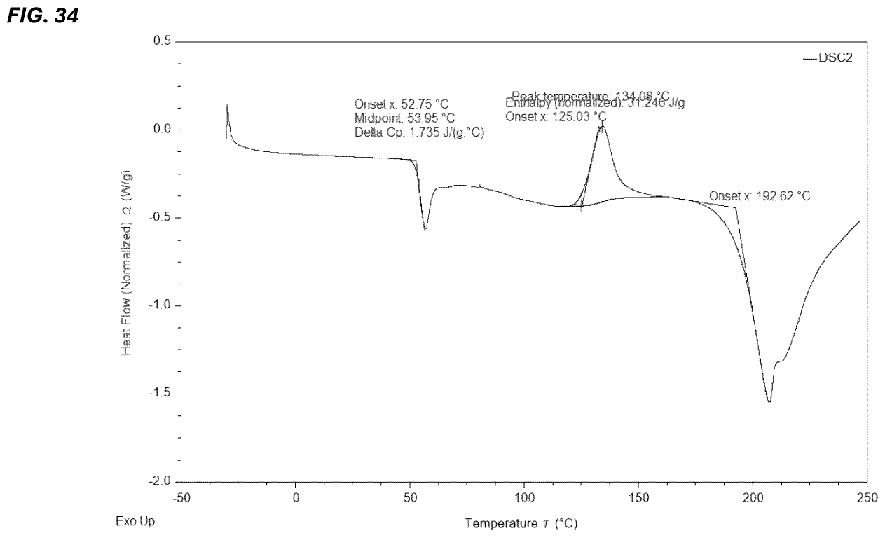

The resulting DSC thermogram displayed a glass transition at 53.95° C. followed by an exothermic event with an onset of 125.03° C. and a broad endothermic event with an onset of 192.62° C. The observation of a glass transition in combination with the XRPD analysis confirmed the amorphous nature of the original material. The observation of the glass transition indicates a change in the sample from a rigid, glassy state to a more flexible state. The subsequent exothermic event (125.03° C.) following the glass transition indicated crystallization of the material followed by an endothermic event indicating melt of the dynamically generated crystalline material during the DSC experiment (FIG. 34). DSC analysis revealed that spray dried Composition 22 is an amorphous solid.

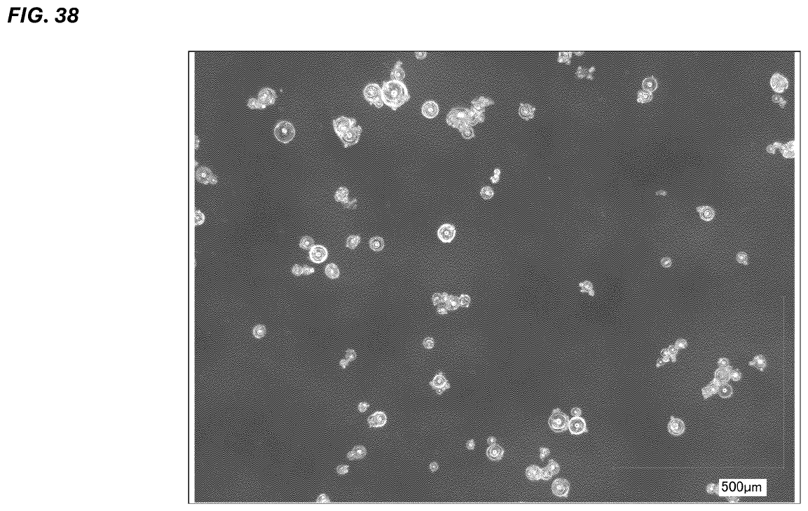

Spray dried Composition 22 was characterized using optical microscopy. The material displayed conglomerates of spherical particles and individual spherical particles of various sizes and shapes (FIG. 38). Data showed that spray dried Composition 22 exists as individual and agglomerated spherical particles, further suggesting that it is an amorphous rather than crystalline material.

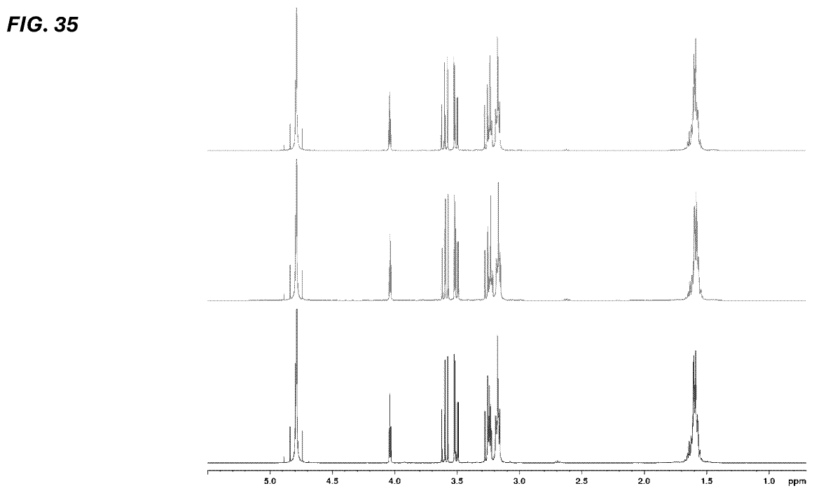

The stacked ¹H NMR spectra of spray dried Composition 22 compared to the spectra of Compositions 14 and 15 are displayed in FIG. 35. The ¹H NMR spectra of three samples exhibit similar chemical shifts and multiplicity indicating similar qualitative composition of the lots. However, the difference in signal intensity observed in specific regions indicates that the relative quantities of components in spray dried Composition 22 differ from those in the other two samples.

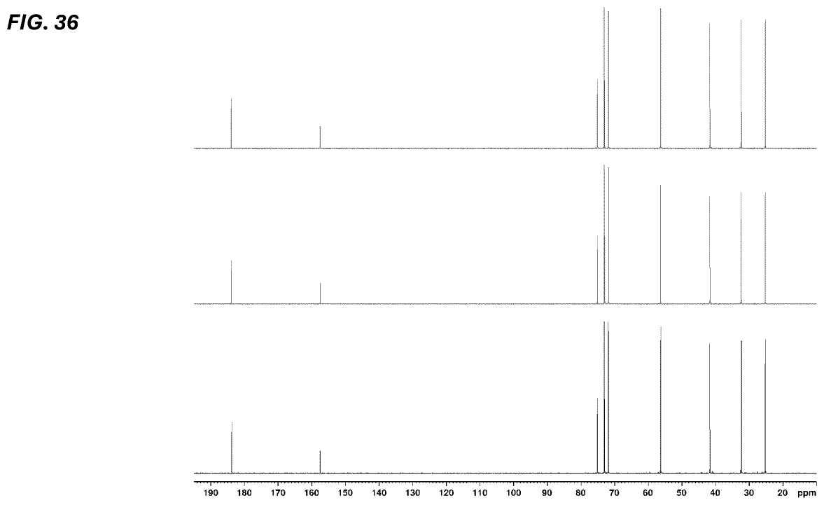

The stacked ¹³C NMR spectra of spray dried Composition 22 compared with freeze dried Compositions 14 and 15 are displayed in FIG. 36. Overall, the ¹³C chemical shifts of the three samples are generally similar, but variations were observed in the carbonyl carbon chemical shifts of arginine across the different samples. Compositions 14 and 15 exhibited carbonyl carbon signals at 183.92 ppm and 183.91 ppm, respectively. Notably, the spray dried Composition 22 displayed a more shielded carbonyl carbon signal at 183.78 ppm. This small difference (0.13˜0.14 ppm) suggests slight changes in the local electronic environment of the carbonyl group.

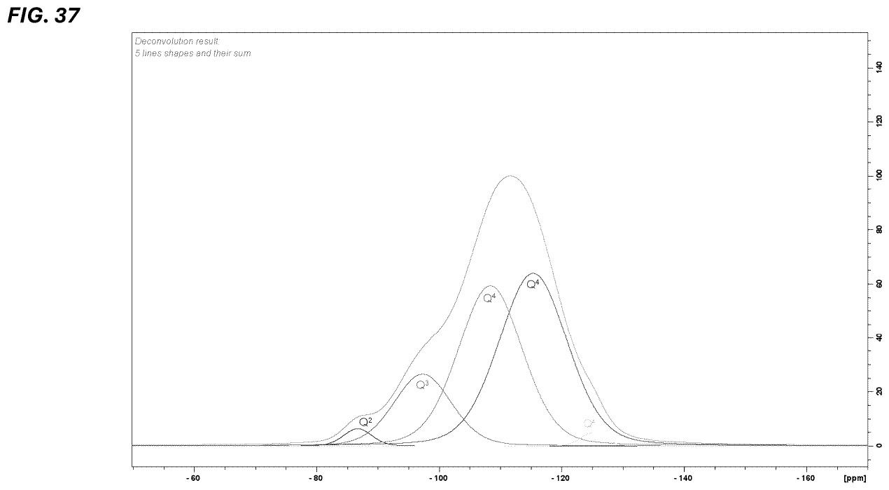

FIG. 37 displays the ²⁹Si ssNMR spectrum of spray dried Composition 22. The chemical shifts, area intensity (integration), and the percentages of the Qⁿ units, including the total Si—OH content of each sample, are summarized in Tables 4 and 5. The reported values are rounded to the second decimal point after the calculations are completed.

TABLE 4
²⁹Si ssNMR chemical shift and area intensity (integration)
for Qⁿ units in various spray dried Composition 22 samples

| Chemical | | | |
|---|---|---|---|
| Shift (ppm) | Area | Rel. Area (%) | ^{Qn} unit |
| −86.7 | 5382 | 1.82% | Q² |
| −97.3 | 44916 | 15.23% | Q³ |
| −108.4 | 112011 | 37.97% | Q⁴ |
| −115.4 | 129058 | 43.75% | Q⁴ |
| −125.2 | 3647 | 1.24% | Q⁴ |

TABLE 5
% of the Qⁿ units and the total Si—OH content

| | | | Total Si—OH |
|---|---|---|---|
| Q²Unit (%) | Q³ Unit (%) | Q⁴ Unit (%) | Content (%) |
| 1.82% | 15.23% | 82.95% | 18.87% |

### Example 23: Preparation of Compositions 23, 24, and 27

Representative procedure to prepare Composition 23 NMR sample: 40.2 mg of Composition 23 was dissolved in 1 mL of D₂O.

Representative procedure to prepare Composition 24 NMR sample: 38.6 mg of milled Composition 24 was dissolved in 1 mL of D₂O.

Compositions 23, 24, and 27 are freeze dried compositions comprising arginine, sodium silicate, inositol, and sodium hydroxide in different quantities.

The individual components of a composition (arginine, inositol, and sodium silicate) and Compositions 21, 23, and 24 were analyzed by diffuse reflectance infrared Fourier transform spectroscopy (DRIFTS). Additionally, a physical mixture of arginine, inositol, and sodium silicate was prepared and analyzed by DRIFTS.

If the composition is a physical mixture of its components, the IR spectrum of the mixture will be similar to a linear combination of the IR spectra of its individual components. In other words, the IR spectral peaks for each component will be present in the IR spectrum of the mixture; no significant frequency shifting will be observed, and no new peaks will be observed. Conversely, if the composition is an electrostatically stabilized complex, peak shifting and/or new peaks will be expected in the IR spectrum of the complex as compared to the spectra of the individual components.

The spectral sum of the composition components was calculated as a linear combination of the spectra of the three individual components. Comparison of the spectral sum and the spectrum of Physical Mixture S indicated that Physical Mixture S was a physical mixture of arginine, inositol, and sodium silicate, as expected.

The spectra of Compositions 21, 23, and 24 were compared to the spectra of Physical Mixture S and the individual components of the composition (FIG. 41). While there was variation in peak intensities between the various composition samples, diagnostic signals from arginine, inositol, and sodium silicate were present in all of the composition samples. Peaks in the region 1475-1720 cm⁻¹ and additional peaks in the region 500-875 cm⁻¹ clearly indicate the presence of arginine in the composition mixtures. While they are of lower intensity, the peaks and/or shoulder peaks at 1052, 929, 898, 892, and 586 cm⁻¹ indicate the presence of inositol. Variations in peak intensities may be due to a sampling bias during preparation of the IR samples, due to the relatively small sample quantity used for DRIFTS samples (1-2 mg), and possible heterogeneity of the powdered composition samples. The presence of a shoulder at 3625 cm⁻¹ indicates the presence of sodium silicate. Full lists of IR peaks for each sample are included in Table 6. The IR spectral data supports the conclusion that the compositions are physical mixtures of its components, rather than an electrostatically stabilized complex. Peaks corresponding to the individual composition components were present in the composition mixtures. No significant peak shifting was observed, and no new peaks were observed, indicating that the chemical environments of the components within the mixtures were consistent with the chemical environments of the components as pure substances.

TABLE 6
IR peak lists for multiple composition samples.

| Physical | Composition | Composition | Composition |
|---|---|---|---|
| Mixture S | 21 | 23 | 24 |
| 433.39 | 551.26 | 487.84 | 551.20 |
| 450.11 | 610.57 | 492.81 | 610.24 |
| 495.72 | 713.40 | 521.70 | 652.43 |
| 550.95 | 769.60 | 551.44 | 710.75 |
| 587.57 | 824.22 | 610.93 | 772.08 |
| 610.09 | 846.65 | 654.11 | 846.83 |
| 711.63 | 881.61 | 712.14 | 881.59 |
| 766.92 | 926.54 | 772.44 | 916.77 |
| 846.70 | 989.43 | 793.11 | 982.67 |
| 891.90 | 1048.65 | 846.68 | 1035.95 |
| 898.23 | 1121.68 | 881.34 | 1049.83 |
| 929.37 | 1139.55 | 916.57 | 1138.28 |
| 1001.02 | 1184.66 | 925.77 | 1184.77 |
| 1050.97 | 1263.18 | 980.68 | 1261.72 |
| 1114.75 | 1332.99 | 1036.27 | 1333.07 |
| 1145.54 | 1377.06 | 1051.24 | 1351.39 |
| 1185.84 | 1420.37 | 1139.09 | 1376.30 |
| 1195.39 | 1441.76 | 1184.82 | 1421.76 |
| 1218.93 | 1450.92 | 1261.51 | 1441.46 |
| 1246.14 | 1474.85 | 1333.00 | 1450.59 |
| 1332.91 | 1566.02 | 1351.31 | 1475.05 |
| 1377.22 | 1628.69 | 1376.32 | 1561.32 |
| 1418.65 | 1677.47 | 1421.67 | 1618.98 |
| 1442.09 | 2864.02 | 1441.44 | 1678.64 |
| 1450.17 | 2944.93 | 1450.55 | 1721.01 |
| 1474.67 | 3172.30 | 1475.00 | 2863.67 |
| 1568.51 | 3298.22 | 1560.25 | 2944.94 |
| 1628.28 | 3358.06 | 1619.32 | 3062.27 |
| 1677.55 | | 1678.28 | 3297.72 |
| 2863.99 | | 1720.99 | 3358.40 |
| 2923.79 | | 2863.74 | |
| 2944.26 | | 2944.87 | |
| 3180.21 | | 3065.93 | |
| 3298.63 | | 3261.74 | |
| 3357.99 | | 3298.24 | |
| | | 3358.27 | |

### Investigation of the Physical State of Several Compositions, Compositions, and Ingredients Thereof Using NMR Spectroscopy

NMR analyses on samples prepared by freeze drying and spray drying were analyzed to examine how sample preparation methods influence physical states. Products generated using different silicates (e.g., sodium silicate vs. potassium silicate) were analyzed to determine the impact of the silicates on the properties of the compositions. In addition, physical mixtures (labeled physical mixtures S and P, using two different types of silicates) were prepared and analyzed to compare to the spectra obtained from the freeze dried samples (Compositions 15, 23, and 24).

### ¹H NMR Analysis of Compositions

To determine whether the compositions are physical mixtures or electrostatically stabilized complexes, ¹H NMR spectroscopy was first employed on various samples and their individual components (e.g., arginine, inositol, omithine). Table 7 shows the acquisition parameters for several compositions.

TABLE 7
Acquisition parameters for NMR spectroscopy

| Parameter | ¹H | | T¹ | | T² | | DOSY | |
|---|---|---|---|---|---|---|---|---|
| Sequence | zg30 | | zgpg | | apt | | zgpg | |
| Size of FID (TD) | 65536 | | 65536 | | 82542 | | 47618 | |
| Acquisition Time | 4 | sec | 1.38 | sec | 1.73 | sec | 1 | sec |
| Spectral width (SW) | 20.48 | ppm | 2.98-6.94 | ppm | 20.48 | ppm | 20.48 | ppm |
| D1 (relaxation | 4 | sec | 15 | sec | 10-12 | sec | 10 | sec |
| delay) | | | | | | | | |
| Number of Scans | 16 | | 8 | | 16 | | 16 | |
| Transmitter | 400.15 | MHz | 400.15 | MHz | 400.15 | MHz | 400.15 | ppm |
| Frequency | | | | | | | | |
| Transmitter | 6.0 | ppm | 3.41-3.97 | ppm | 6.5 | ppm | 6.5 | ppm |

Frequency Offset
(O1P)

If the composition sample is a physical mixture of its components, distinct signals for each component, no significant chemical shift perturbation (CSP), and no new peaks indicative of new chemical environments (such as complex formation) would be present. If the f composition exists as an electrostatically stabilized complex, CSP, peak broadening, disappearance, or merging of signals will be observed.

The ¹H NMR spectra of arginine and inositol are consistent with those of the reference spectra, indicating that the materials analyzed are indeed arginine and inositol. In addition, the ¹H NMR spectra of inositol and arginine exhibit unique ¹H peaks that can be used to identify them in the compositions samples or in physical mixtures, except for some overlapping signals near 3.27 ppm.

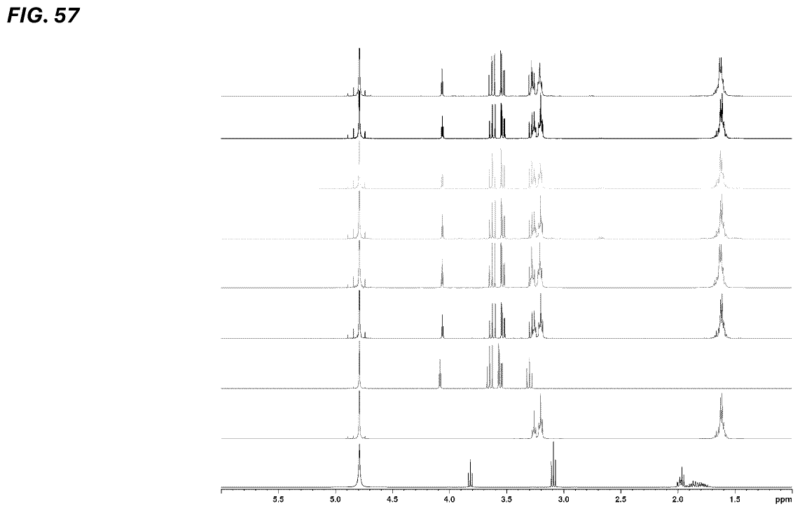

The acquired ¹H NMR spectra of the physical mixtures and compositions samples are compared to the spectra of inositol and arginine in FIG. 57, which shows that all of the compositions products and physical mixtures contain both inositol and arginine, and each component can be clearly identified except for the overlapping signals near 3.27 ppm. Based on the comparative analysis between various compositions NMR spectra, a minor upfield shift of approximately 0.02 ppm was observed in the inositol's ¹H chemical shifts in compositions/physical mixture relative to those observed in pure inositol. The observation of comparable inositol ¹H chemical shifts in the compositions and physical mixtures indicates that similar complexation occurs in both sample types. In the case of arginine in the compositions, samples formulated with Na-silicate exhibited no observable CSP and peak broadening, whereas those containing K-silicate displayed slight CSP and slight peak broadening. This phenomenon may be attributed to differences in ionic radius, coordination behavior, solvation characteristics, and binding strength between potassium and sodium silicates, which require further investigation. It is noted that there is a slight difference between spray dried Composition 22 and the physical mixture P near 3.27 ppm.

### Example 24: Preparation of Physical Mixture S for IR and NMR Sampling

IR: Physical Mixture S was prepared by combining 200.4 mg of arginine, 103.4 mg of inositol, 91.2 mg of sodium silicate solids, and 11.7 mg of sodium hydroxide in an agate mortar and pestle. The mixture was ground to a fine, well-mixed powder using the mortar and pestle. A portion of the sample was diluted approximately 50:1 by mass in KBr and analyzed by DRIFTS.

NMR: 200.2 mg of L-Arginine, 103.4 mg of Inositol, 90.9 mg of Sodium Silicate, and 11.6 mg of NaOH were combined in a 20 mL glass vial, followed by adding 10 mL of D₂O. The solution was sonicated for 30 minutes and vortexed for 20 minutes. This procedure was performed twice, with an additional hour of sonication.

### Example 25: Preparation of Physical Mixture P for NMR Sampling

199.9 mg of L-Arginine, 103.3 mg of Inositol, and 336.5 mg of Potassium Silicate were combined in a 20 mL glass vial, followed by adding 7 mL of water. The solution was vortexed for 2-3 minutes, then freeze dried overnight. 39.1 mg of the dried material was dissolved in 1 mL of D₂O.

### Example 26: Characterization of Milled Composition 25 and Admixture 26

### Particle Size Distribution (PSD) Analysis

The PSDs are summarized in Table 8.

TABLE 8
PSD results for additional samples

| | Additional | | | |
|---|---|---|---|---|
| Composition # | Information | d₁₀ (μm) | d₅₀ (μm) | d₉₀ (μm) |
| Admixture 26 | Sample 1 | 21.9 | 121 | 424 |
| | Sample 2 | 21.7 | 115 | 391 |
| | Sample 3 | 22.0 | 120 | 432 |
| | Composite | 21.9 | 120 | 430 |
| Inositol | | 27.5 | 125 | 383 |
| Milled Composition 25 | Sample 1 | 25.5 | 189 | 579 |
| | Sample 2 | 26.2 | 191 | 559 |
| | Sample 3 | 24.9 | 192 | 580 |
| | Composite | 23.8 | 186 | 554 |

The PSD results for milled Composition 25 were consistent amongst the analyzed lots. The average Dv10, Dv50, and Dv90 were ˜25, 190, and 569 μm, respectively. All analyses had RSDs within USP tolerance. The PSD results for admixture 26 were also consistent amongst the analyzed lots, with a Dv10 of ˜22 μm, a Dv50 of ˜119 μm, and a Dv90 of ˜419 μm. Smaller particles of ˜1 μm were observed in Samples 1 and 3 of admixture 26, and the composite material. No particles were observed between 1-2 μm (i.e., particles of ˜1 μm and particles between ˜2-1000 μm). The observed PSD of admixture 26 was consistent with the expected results, considering that it is a physical mixture of milled Composition 25 and inositol. The PSD of inositol yielded a Dv10 of 27.5 μm, a Dv50 of 125 μm, and a Dv90 of 383 μm.

### Example 27: Preparation of Composition 28

Sodium silicate (as 9.1 wt % Na₂O and 29.0% SiO₂) was frozen and freeze dried with the following parameters to afford lyophilized Na Silicate: Drying temperature (180° F.); Pressure (100-150 mtor); Drying Time (25 h). Lyophilized Na Silicate (100 mg) was added to water (10 g) (1 wt %) and sonicated for 30 min. The material did not dissolve and contained sedimentation.

Arginine (200 mg), inositol (100 mg), and lyophilized sodium silicate (100 mg) were combined in a 20 mL glass vial to form Composition 28, followed by adding 8 g of water (5 wt %). The solution was sonicated for 30 minutes and vortexed for 20 minutes. This physical mixture was not completely dissolved. The solution is cloudy and has sedimentation.

### Example 28: Preparation of Composition 29

Potassium silicate (as 8.3% K₂O and 28.9% SiO₂) was frozen and freeze dried with the following parameters to afford lyophilized K Silicate: Drying temperature (180° F.); Pressure (100-150 mtor); Drying Time (25 h). Lyophilized K Silicate (100 mg) was added to water (10 g) (1 wt %) and sonicated for 30 min. The material did not dissolve and contained sedimentation.

Arginine (200 mg), inositol (100 mg), and lyophilized sodium silicate (100 mg) were combined in a 20 mL glass vial to form Composition 28, followed by adding 8 g of water (5 wt %). The solution was sonicated for 30 minutes and vortexed for 20 minutes. This physical mixture was not completely dissolved. The solution is cloudy and has sedimentation.

### Example 29: Formulation for Milled Compositions 25 Beverage

### Solution Preparation

Water is charged to a vessel. Milled composition 25 was added and agitated until dissolved. The following ingredients were added in order and incorporated: acids, potassium citrate, sucralose, and flavor. The ingredients and serving amounts are shown in Table 9 below.

TABLE 9
Milled Composition 25 ingredients and serving amounts

| | | % |
|---|---|---|
| | per Serving | per |
| Ingredient | grams | Serving |
| Water | 350.1150 | 98.6239 |
| Milled Composition 25 | 1.5000 | 0.4225 |
| Malic Acid | 1.2000 | 0.3380 |
| Citric Acid | 0.6500 | 0.1831 |
| Potassium Citrate | 0.3000 | 0.0845 |
| Sucralose | 0.0600 | 0.0169 |
| Cranberry Fl | 0.5500 | 0.1549 |
| Hibiscus Fl | 0.5500 | 0.1549 |
| Orange FL | 0.0750 | 0.0211 |

### Batching Protocol

A tank was filled with 70% of required water per batch requirement. The concentrate was added. The tank was kept under continuous mixing/agitation while ingredients were added. The remaining water was added to the tank to meet batch requirements. The batch was evaluated to ensure specifications are met. The tank was then chilled to >35° F., followed by de-aeration and carbonation to 2.2 atm. Cans were filled in accordance with the Standard Operating Procedure and pasteurized in accordance with process authority letter.

### Example 30: Formulation for Admixture 26 Beverage

### Solution Preparation

Water is charged to a vessel. Admixture 26 was added and agitated with an immersion blender until dissolved. The following ingredients were added in order and incorporated: acids, potassium citrate, sucralose, and flavor. The ingredients and serving amounts are shown in Table 10 below.

TABLE 10
Admixture 26 ingredients and serving amounts

| | per Serving | % |
|---|---|---|
| Ingredient | grams | per Serving |
| Water | 349.3900 | 98.4197 |
| Admixture 26 | 1.6000 | 0.4507 |
| Malic Acid | 1.1500 | 0.3239 |
| Citric Acid | 0.6000 | 0.1690 |
| Potassium Citrate | 0.3000 | 0.0845 |
| Sucralose | 0.0600 | 0.0169 |
| Cranberry Fl | 1.0000 | 0.2817 |
| Hibiscus Fl | 0.8500 | 0.2394 |
| Orange FL | 0.0500 | 0.0141 |

### Batching Protocol

A tank was filled with 70% of required water per batch requirement. The concentrate was charged. The tank was kept under continuous mixing/agitation while ingredients were added. The remaining water was added to the tank to meet batch requirements. The batch was evaluated to ensure specifications are met. The tank was then chilled to >35° F. Cans were filled in accordance with the Standard Operating Procedure and pasteurized in accordance with process authority letter.

### Instrumental Techniques

### XRPD (for Compositions 1, 2, and 3)

XRPD patterns were collected with a PANalytical X'Pert PRO MPD or Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and an antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5. The data acquisition parameters for each pattern are displayed above the image in the Data section of this report including the divergence slit (DS) before the mirror.

### XRPD for L-Arginine and Inositol Reference Standards and Composition 13

The samples were prepared in silicon low background holders using light manual pressure to keep the sample surface flat and level with the reference surface of the sample holder. The single crystal Si low background holder has a circular recess (10 mm diameter and about 0.2 mm depth) that holds the sample.

The Rigaku Smart-Lab diffraction system was configured for Bragg-Brentano reflection geometry using a line source X-ray beam. The Bragg-Brentano geometry was controlled by passive divergence and receiving slits with the sample itself acting as the focusing component for the optics. Data collection parameters are shown in Table 11. The plots were generated using PlotMon V2.1.1.0 software.

TABLE 11
Data Collection Parameters

| Parameter | Value | Parameter | Value |
|---|---|---|---|
| Geometry | Bragg-Brentano | Receiving Slit 1 (mm) | 18 |
| Tube Anode | Cu Kα | Receiving Slit 2 (mm) | 20.1 |
| Tube Type | Long Fine Focus | Start Angle (°2θ) | 2 |
| Tube Voltage (kV) | 40 | End Angle (°2θ) | 40 |
| Tube Current (mA) | 50 | Step Size (°2θ) | 0.02 |
| Detector | HyPix3000 | Scan Speed (°2θ/min) | 6 |
| Monochromatization | Kβ Filter | Spinning (rpm) | 11 |
| Incident Slit (°) | 1/3 | Sample Holder | Large well silicon low |
| | | | background holder |

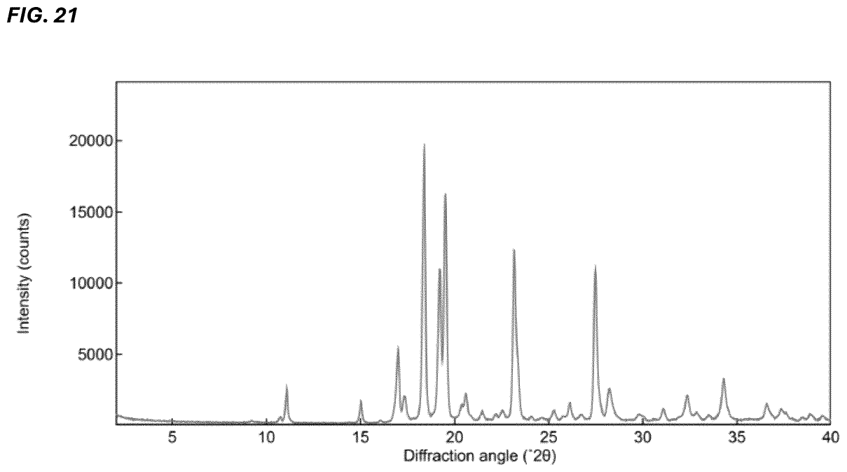

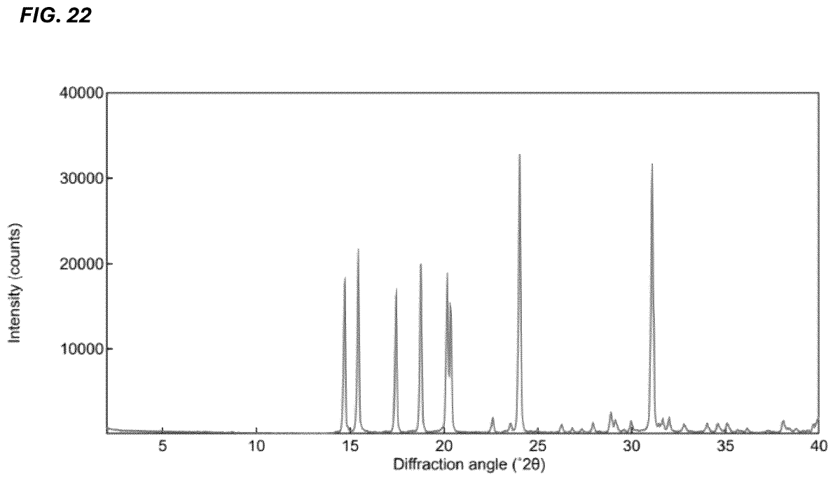

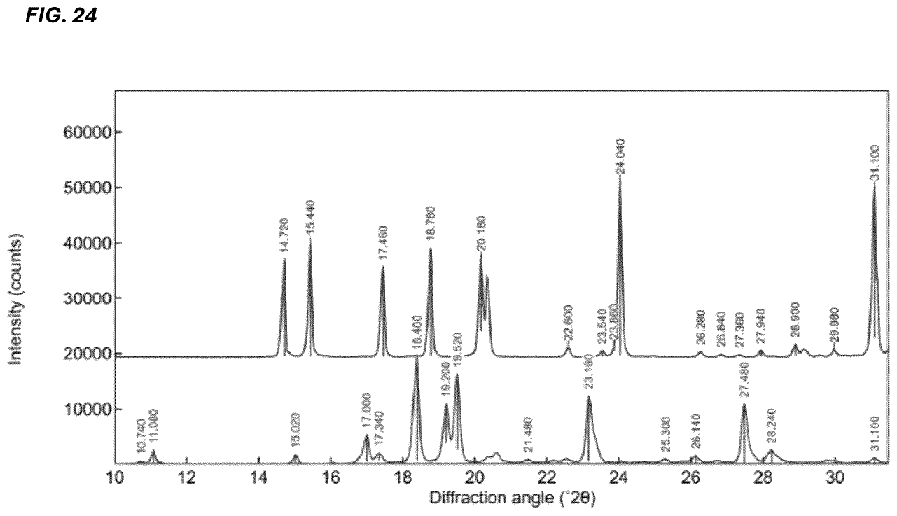

XRPD patterns for reference standards L-arginine and inositol are shown in FIGS. 21 and 22, respectively. An XRPD pattern of Composition 13 is shown in FIG. 23. FIG. 24 is stack plot of XRPD patterns of inositol and L-arginine with peak labels.

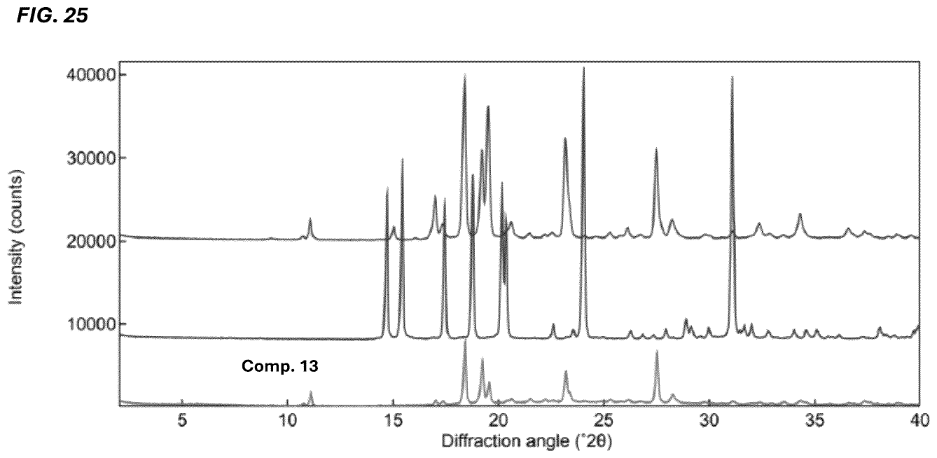

FIG. 25 is stacked plot of XRPD patterns of Composition 13, inositol and L-arginine.

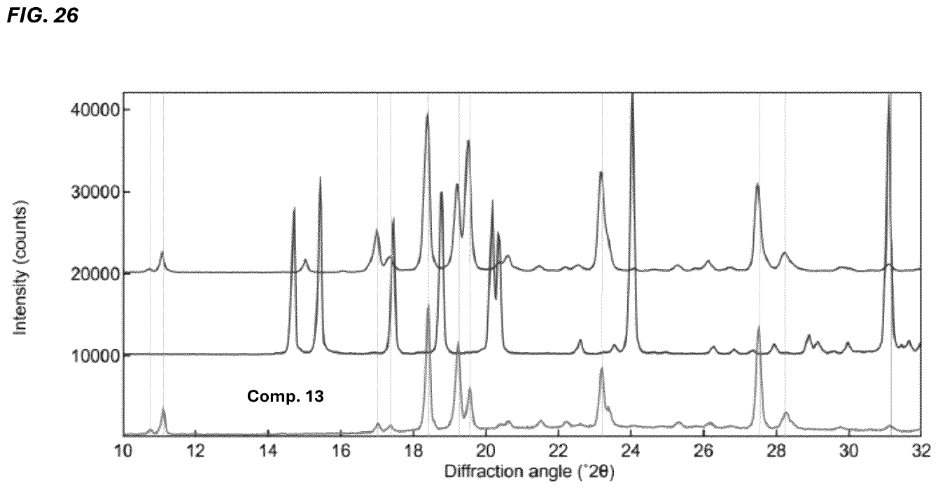

FIG. 26 is stacked plot of XRPD patterns of Composition 13, inositol and L-arginine with the apex of the XRPD peaks for Composition 13 marked with lines to show the alignment with the reference standards.

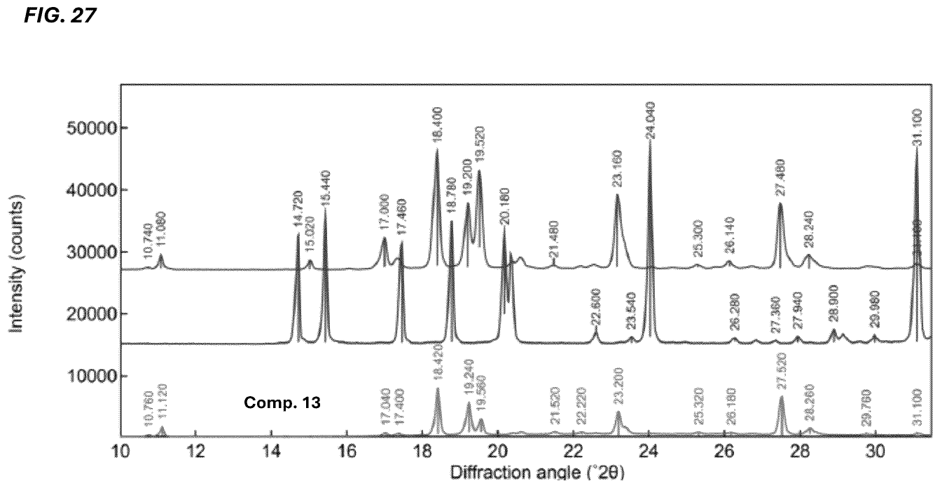

FIG. 27 is stacked plot of XRPD patterns of Composition 13, inositol and L-arginine with peak labels.

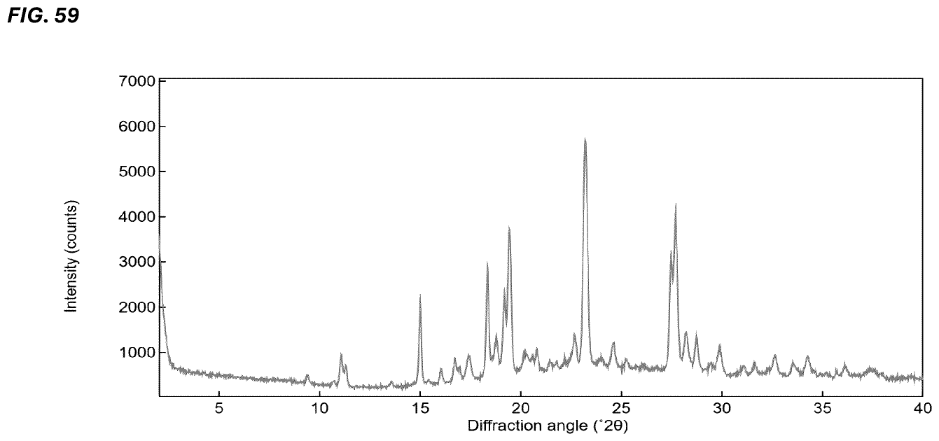

FIG. 59 is an XRPD pattern of Composition 27. Additional peaks at ˜9.41, 11.34, 13.60, 15.03, 16.09, 16.72, 18.73, 22.74, 27.72, 28.79, 29.42, 29.45, 31.68, 32.61, and 36.10° 2θ observed in the XRPD pattern of Composition 27 indicates a potential solid-state form change compared to Composition 13.

For spray dried Composition 22 and Compositions 20 and 21, the peak list, intensity, and FWHM were generated using SmartLab Studio II software, and the amorphous baseline was subtracted for the list generation. Data collection parameters are shown in Table 12.

TABLE 12
Data Collection Parameters

| Parameter | Value | Parameter | Value |
|---|---|---|---|
| Geometry | Bragg-Brentano | Receiving Slit 1 (mm) | 18 |
| Tube Anode | Cu Kα | Receiving Slit 2 (mm) | 20.1 |
| Tube Type | Long Fine Focus | Start Angle (°2θ) | 2 |
| Tube Voltage (kV) | 40 | End Angle (°2θ) | 40 |
| Tube Current (mA) | 44 | Step Size (°2θ) | 0.02 |
| Detector | D/teX Ultra 250 | Scan Speed (°2θ/min) | 2 |
| Monochromatization | Kβ Filter | Spinning (rpm) | 11 |
| Incident Slit (°) | 1/3 | Sample Holder | Large well silicon low |
| | | | background holder |

### TGA

TGA analysis of compositions 1-4 was conducted using a TA Instruments Q500 TGA analyzer. Samples were heated in nitrogen at 20° C./minute to 100° C., at which point the ramp rate was slowed to 10° C./minute. Above 600° C., oxygen was introduced, and the sample further heated at the same ramp rate to 800° C. The software incorporates a high-resolution feature which slows the ramp rate when weight change is detected, improving the resolution of components.

The TGA analysis for spray dried Composition 22 was conducted using a TA Instruments Q5500 Discovery Series instrument. The instrument balance was calibrated using class M weights, and the temperature was calibrated using alumel. The nitrogen purge was ˜10 mL per minute at the balance and ˜25 mL per minute at the furnace. The sample was placed into a pre-tared platinum pan and heated from approximately 25° C. to 300° C. at 10° C./minute. The instrument was controlled, and the data was analyzed using Trios v5.0.0.44608.

### Differential Scanning Calorimetry (DSC) Measurements

DSC analysis was performed using a Mettler-Toledo TGA/DSC3+ analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum/ceramic pan configured as the sample pan was placed on the reference platform. The furnace was heated under nitrogen. The data acquisition parameters for the thermogram are displayed on the image.

DSC analysis for spray dried Composition 22 was carried out using a TA Instruments Q2000 instrument. The instrument temperature calibration was performed using indium. During analysis, the DSC cell was kept under a nitrogen purge of ˜50 mL per minute. The sample was placed in a standard, crimped aluminum pan and was heated from approximately −30° C. to 250° C. at a rate of 10° C. per minute.

### Scanning Electron Microscopy (SEM) Experiments

Scanning electron microscopy (SEM) was conducted on each of the samples using a Nanoscience Phenom ProX SEM analyzer. Each sample was sputter coated with gold prior to analysis. Images of each sample were taken at 500×, 1000×, 2000×, 5000×, and 10000× magnification.

The solubility of certain exemplary compositions of the present disclosure is summarized in Table 13.

TABLE 13
Solubility of Exemplary Compositions

| | | | | | | | | | Dissolution time |
|---|---|---|---|---|---|---|---|---|---|
| | 1.6 | 2.5 | 5 | 10 | 15 | 20 | 25 | 50 | (10 wt % solutions |
| Entry | wt % | wt % | wt % | wt % | wt % | wt % | wt % | wt % | only) |
| Composition 1 | PS | PS | PS | PS | PS | PS | NT | NT | NT |
| Composition 2 | PS | PS | PS | PS | PS | PS | NT | NT | NT |
| Composition 3 | PS | PS | PS | PS | PS | PS | NT | NT | NT |
| Composition 4 | PS | PS | PS | PS | PS | PS | NT | NT | NT |
| Composition 5 | S | S | S | PS | PS | PS | NT | NT | NT |
| Composition 6 | S | S | S | S | S | S | NT | NT | NT |
| Composition 7 | S | S | S | S | S | S | NT | NT | NT |
| Composition 8 | S | S | S | S | S | S | NT | NT | NT |
| Composition 9^{§} | NT | NT | NT | NT | NT | NT | NT | S | NT |
| Composition 10^{§} | NT | NT | NT | NT | NT | NT | NT | S | NT |
| Composition 11^{§} | NT | NT | NT | NT | NT | NT | S | NT | NT |
| Composition 12 | PS | PS | PS | PS | PS | PS | NT | NT | NT |
| Composition 13 | PS | PS | PS | PS | PS | PS | NT | NT | NT |
| Composition 14 | PS | PS | PS | PS | PS | PS | NT | NT | PS @ 40 min |
| Composition 15 | PS | PS | PS | PS | PS | PS | NT | NT | PS @ 20 min |
| Composition 16 | S | S | S | S | S | S | NT | NT | S @ 6 min 12 s |
| Composition 17 | S | S | S | S | S | S | NT | NT | S @ 4 min 55 s |
| Composition 18 | PS | PS | PS | PS | PS | PS | NT | NT | PS @ 30 min |
| Composition 19 | PS | PS | PS | PS | PS | PS | NT | NT | PS @ 20 min |
| Composition 20 | S | S | S | S | S | S | NT | NT | S @ 6 min 9 s |
| Composition 21 | S | S | S | S | S | S | NT | NT | S @ 4 min 15 s |
| Composition 28 | NT | NT | PS | NT | NT | NT | NT | NT | NT |
| Composition 29 | NT | NT | PS | NT | NT | NT | NT | NT | NT |

PS: Partially soluble at tested concentration: sedimentation and/or particulates are observed by visual inspection.
S: Soluble at tested concentration: no sedimentation or particulates are observed by visual inspection.
NT: Not tested
§ —Refer to Example section for solution preparations.

### Optical Microscopy

Optical Microscopy (OM) for was conducted using a Keyence VHX-2000E digital microscope equipped with a VH-Z20R variable magnification lens (20-200×). Samples were placed onto the automatic stage and illuminated with reflected light. Photomicrographs were acquired and processed using the VHX communication v2.35 software.

### NMR Spectroscopy

The NMR spectroscopic analyses were conducted using a Bruker NEO 400 MHz spectrometer at Triclinic Labs. For ¹H and ¹³C solution-state NMR measurements, a 5 mm PABBO probe was employed, while ²⁹Si single-pulse (SP) magic-angle-spinning (MAS) NMR spectra were acquired using a 4 mm iProbe. Data processing was performed with TopSpin GxP 4.1.4 software. The ¹H chemical shift was referenced to the chemical shift of the residual solvent peak (e.g., D₂O at 4.79 ppm) and the ¹³C chemical shift was externally referenced to the ¹³C signal of C₆D₆ at 128.4 ppm. The ²⁹Si chemical shift was externally referenced to the ²⁹Si signal of trimethylsilane (TMS) at 0 ppm. More detailed NMR acquisition parameters are provided in Tables 5. Spectral deconvolution of ²⁹Si ssNMR spectra was performed utilizing the deconvolution function in Topspin 4.1.4 software to deconvolve overlapping peaks in the spectra. The NMR spectra were deconvoluted into individual peaks of Gaussian-Lorentzian functions, corresponding to the Qn units of the samples. Table 14 shows the acquisition parameters for spray dried Composition 22.

TABLE 14
Acquisition parameters for NMR spectroscopy

| Parameter | ¹H NMR | | ¹³C NMR | | ²⁹Si ssNMR | |
|---|---|---|---|---|---|---|
| Sequence | zg30 | | zgpg | | zg | |
| Size of FID (TD) | 65536 | | 65536 | | 3072 | |
| Acquisition Time | 4 | sec | 1.4 | sec | 38.7 | ms |
| Spectral width | 8197 | Hz | 23810 | Hz | 499.2 | ppm |
| D1 (relaxation delay) | 4 | sec | 5 | sec | 20 | sec |
| Number of Scans | 64 | | 1536 | | 3072 | |
| Transmitter Frequency | 400.15 | MHz | 100.63 | MHz | 79.49 | MHz |
| Transmitter Frequency | 6.0 | ppm | 110.0 | ppm | −110 | ppm |
| Offset (O1P) | | | | | | |
| Line broadening | 0.1 | Hz | 1.0 | Hz | −80 | Hz |

### IR Spectroscopy

IR spectra were acquired using a Thermo Scientific model iS50 Fourier-transform (FT) IR spectrophotometer equipped with a deuterated triglycine sulfate (DTGS) detector, a potassium bromide (KBr) beamsplitter, and a Polaris™ long-life IR source. A diffuse reflectance sampling accessory with a spectral range of 4000-400 cm⁻¹ was used to collect data. Each sample was prepared by diluting approximately 50:1 by mass in KBr and grinding the mixture into a fine powder using a mortar and pestle. Each diluted sample was packed into a micro sample cup with a smooth, level surface. Signal acquisition was optimized using a sample accessory mirror and adjusting the stage height to maximize the peak-to-peak value. The single beam scans of the background (air) and sample were collected with 128 signal averaged scans at a resolution of 2 cm⁻¹ over the spectral range of 4000-400 cm⁻¹. The accessory mirror previously used for system optimization was also used to collect the background. A background was collected before every sample spectrum, and the sample chamber was allowed to purge with nitrogen gas for approximately 5 minutes after each instance of opening the sample compartment. Sample spectra were automatically calculated and presented in Log(1/R) units. The wavelength calibration was verified using a certified polystyrene standard. Data acquisition and processing were performed using OMNIC v9.11 software.

### Particle Size Distribution

The particle size analysis of milled Composition 25 sample was performed using a Malvern Master-Sizer 3000 model MAZ3000 equipped with the Hydro MV dispersion accessory for wet analysis. The instrument performance is verified with a NIST-traceable polydisperse standard containing particles in the range of 10-100 μm (Whitehouse Scientific, Catalog number PS314). The MS3000 has a PSD range of 0.01-1500 μm for wet analysis. The instrument was configured with shortened feed lines from the circulatory reservoir to the measurement cell. The parameters in Table 15 were implemented. Repeatability was assessed. The results demonstrated that consistent results were obtained across the Dv10, Dv50, and Dv90, confirming the % RSD within USP tolerance in USP <429>.

TABLE 15
Final laser diffraction size analysis

| | |
|---|---|
| Instrument | Malvern MasterSizer 3000 |

| Dispersion Accessory | Hydro MV | |
|---|---|---|
| Refractive Index | 1.47 | |
| Absorption Index | 0.01 | |
| Dispersant | Methanol | |
| Dispersant Refractive Index | 1.33 | |
| Stirring Speed | 2000 | rpm |
| Ultrasound | None | |
| Background Measurement Time | 20 | seconds |
| Sample Measurement Time | 20 | seconds |
| Obscuration | 10-20% | |
| Records per Preparation | 5 | |
| Hold Time | 90 | seconds |

| | |
|---|---|
| Fine Powder Mode | Not Selected |
| Analysis Model | General Purpose |
| Optical Model | MIE Theory |

The aforementioned method was implemented for the additional submitted samples.

### Particle Size Analysis (PSA) Method Development for Milled Composition 25 and Admixture 26

A method for the particle size analysis (PSA) of Milled Composition 25 was developed by laser diffraction. The developed method was used to analyze another composition, i.e., inositol, and the final product, admixture 26, which is a combination of milled Composition 25 fortified with inositol.

### Optical Microscopy

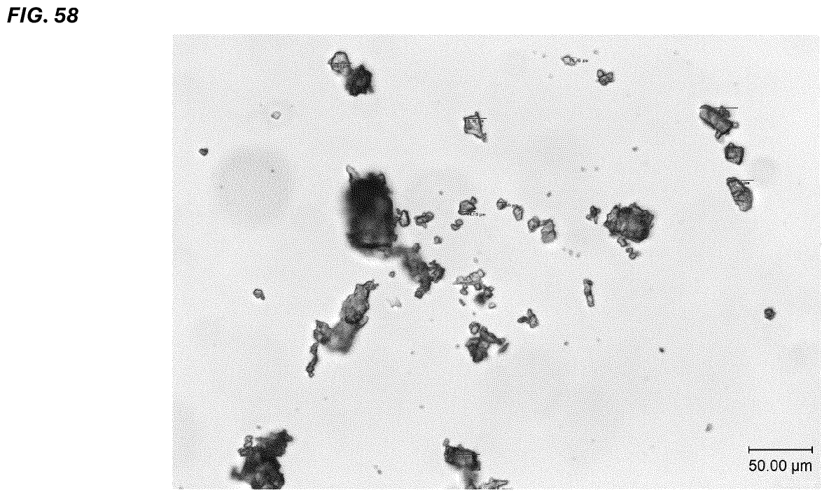

Admixture 26 was examined by optical microscopy (OM) as an orthogonal approach to the laser diffraction PSA technique. Optical microscopy was performed using a Leica DM2500P polarizing microscope equipped with a PAXcam3 digital camera. The microscope's performance was verified with a certified MRS-3XY standard. Samples were prepared on a glass microscope slide with a glass cover slip. The sample was dispersed in silicon oil via capillary action. The slide was then placed on the stage under 4×, 10×, and 20× objectives, and illuminated with transmitted light. The OM image is presented in FIG. 58. The image demonstrated that the sample consisted of primary particles (irregularly shaped) between ˜1 μm and 1085 μm.

### Solution Preparation

Representative procedure to prepare a 1.6 wt % solution: The powdered composition (1.5 g) is charged into a beaker equipped with a stir bar. Water (90 g) is added to the beaker and the solution is stirred for 10 minutes at 20° C. at 500 rpm. The stir bar is removed and the solution visually inspected for sediment and suspended particulates.

Representative procedure to prepare a 2.5 wt % solution: The powdered composition (2.5 g) is charged into a beaker equipped with a stir bar. Water (97.5 g) is added to the beaker and the solution is stirred for 10 minutes at 20° C. at 500 rpm. The stir bar is removed and the solution visually inspected for sediment and suspended particulates.

Representative procedure to prepare a 5 wt % solution: The powdered composition (5 g) is charged into a beaker equipped with a stir bar. Water (95 g) is added to the beaker and the solution is stirred for 10 minutes at 20° C. at 500 rpm. The stir bar is removed and the solution visually inspected for sediment and suspended particulates.

Representative procedure to prepare a 10 wt % solution: The powdered composition (10 g) is charged into a beaker equipped with a stir bar. Water (90 g) is added to the beaker and the solution is stirred for 10 minutes at 20° C. at 500 rpm. The stir bar is removed and the solution visually inspected for sediment and suspended particulates.

Representative procedure to prepare a 15 wt % solution: The powdered composition (15 g) is charged into a beaker equipped with a stir bar. Water (85 g) is added to the beaker and the solution is stirred for 10 minutes at 20° C. at 500 rpm. The stir bar is removed and the solution visually inspected for sediment and suspended particulates.

Representative procedure to prepare a 20 wt % solution: The powdered composition (20 g) is charged into a beaker equipped with a stir bar. Water (80 g) is added to the beaker and the solution is stirred for 10 minutes at 20° C. at 500 rpm. The stir bar is removed and the solution visually inspected for sediment and suspended particulates.

### Sample Preparation

Representative procedure to prepare L-Arginine NMR sample: 38.8 mg of L-Arginine was dissolved in 1 mL of D₂O
Representative procedure to prepare Inositol NMR sample: 34.0 mg of Inositol was dissolved in 1 mL of D₂O

### INCORPORATION BY REFERENCE

All publications and patents mentioned herein are hereby incorporated by reference in their entirety. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1-90. (canceled)

91. A composition comprising:
arginine;
a silicate; and
inositol;
wherein the solubility of the composition in water is at least 5 wt % at 20° C.

92. The composition of claim 91, wherein the composition further comprises a hydroxide source selected from sodium hydroxide or potassium hydroxide.

93. The composition of claim 91, wherein the composition dissolves in water in less than 15 minutes at 5 wt %.

94. The composition of claim 91, wherein the composition is formed by spray drying or freeze drying.

95. The composition of claim 91, wherein the silicate comprises sodium silicate or potassium silicate.

96. A freeze dried composition comprising:
arginine;
a silicate; and
inositol;
wherein the solubility of the composition in water is at least 5 wt % at 20° C.

97. The composition of claim 96, wherein the solubility of the composition in water is at least 5%, at least 10 wt %, at least 15%, at least 20 wt %, at least 30 wt %, at least 40 wt %, or at least 50 wt % at 20° C.

98. The composition of claim 96, wherein the composition further comprises a hydroxide source selected from sodium hydroxide or potassium hydroxide.

99. The composition of claim 98, wherein the hydroxide source is present in a molar ratio from about 0.1:1 to about 0.5:1 relative to the arginine.

100. The composition of claim 96, wherein the silicate comprises sodium silicate or potassium silicate.

101. A composition, comprising:
arginine;
a silicate; and
inositol,
wherein the composition has an X-ray powder diffraction (XRPD) pattern comprising 2-Theta peaks at 18.4±0.1°, 19.2±0.1°, and 27.5±0.1°.

102. The composition of claim 101, wherein the XRPD pattern further comprises a 2-Theta peak at 23.2±0.1° and at 11.1±0.1°.

103. The composition of claim 101, having an X-ray powder diffraction (XRPD) pattern substantially the same as shown in FIG. 23.

104. The composition of claim 91, wherein the arginine, the silicate, and the inositol do not form an electrostatic or ionic complex.

105. The composition of claim 91, wherein the arginine, the silicate, and the inositol form a heterogeneous composition.

106. The composition of claim 91, wherein the composition further comprises a nitrate or a salt thereof, beetroot, or beetroot extract.

107. A method of making a composition comprising:
optionally, arginine;
a silicate; and
inositol;
wherein the solubility of the composition in water is at least 5 wt % at about 20° C.;
the method comprising:
combining the silicate, the inositol, and when present, the arginine in water to form a first mixture;
optionally adding a hydroxide source to the first mixture to afford a second mixture; and
freeze drying the first mixture or the second mixture to afford the composition.

108. The method of claim 107, wherein the solubility of the composition in water is at least 20 wt %, at least 30 wt %, at least 40 wt %, or at least 50 wt % at 20° C.

109. The method of claim 107, wherein the hydroxide source comprises sodium hydroxide or potassium hydroxide.

110. The method of claim 107, wherein the hydroxide source is present in a molar ratio from about 0.1:1 to about 0.5:1 relative to the arginine.

111. The method of claim 107, wherein the silicate comprises sodium silicate or potassium silicate.

112. The method of claim 107, further comprising milling the composition to produce a milled composition.

113. The method of claim 107, further comprising adding additional inositol to the milled composition to produce an admixture.

114. The composition of claim 91 formulated as beverage.

115. The composition of claim 114, wherein the beverage has a pH of about 2 to about 4.

116. The composition of claim 115, wherein the pH is adjusted with citric acid or malic acid.

117. The composition of claim 114, wherein the beverage further comprises a nitrate or a salt thereof, beetroot, beetroot extract, or a combination thereof.

118. The composition of claim 114, wherein the beverage is carbonated or non-carbonated.

119. The composition of claim 114, wherein the beverage has a volume from about 1 oz to about 16 oz.
